(19) United States
(12) Patent Application Publication
(10) Pub. No.: US 2026/0321420 A1
(43) Pub. Date: Oct. 15, 2026

(54) METHOD FOR ASSISTING IN REMEDYING SLEEP PROBLEM

(71) Applicants: Akiyoshi SHIMURA, Tokyo (JP); CHILDREN & FUTURE CO., LTD., Tokyo (JP)

(72) Inventors: Akiyoshi SHIMURA, Tokyo (JP); Yuusaku AKATSUKA, Tokyo (JP)

(21) Appl. No.: 19/462,049

(22) Filed: Jan. 28, 2026

Related U.S. Application Data
(62) Division of application No. 18/846,047, filed on Jan. 24, 2023, now abandoned.

(30) Foreign Application Priority Data
Mar. 11, 2022 (JP) 2022-037715

(51) Int. Cl. G16H 50/20; G16H 10/20; G16H 10/60; G16H 20/70

(52) U.S. Cl. CPC G16H 50/20; G16H 10/20; G16H 10/60; G16H 20/70

(57) ABSTRACT

A method is made for providing solutions for determining sleep disorders and identifying the causes of sleep problems based on the relationship between the lifestyle and sleep, and remedying the sleep problems. The method includes identifying the type of a user's sleep problem by monitoring a sleep duration and sleep schedule, monitoring sleep quality and a daytime function; selecting candidate causes of the type of the sleep problem using a table showing a relationship between types of the sleep problems and suspected causes; making the final determination of the causes of sleep problem; prioritizing lifestyle items using a table showing standardized coefficients of an impact of the lifestyle on the sleep problems for each type of sleep problem separately; and providing countermeasures to the user based on the standardized coefficients in descending order, thereby assisting in remedying the sleep problem of the user.

### CROSS-REFERENCE TO RELATED APPLICATIONS

This application is a divisional application of U.S. patent application Ser. No. 18/846,047, filed Sep. 11, 2024, which is the U.S. National Phase under 35 U.S.C. § 371 of International Application PCT/JP2023/002053, filed Jan. 24, 2023, which claims priority to Japanese Patent Application No. JP2022-037715, filed Mar. 11, 2022, each disclosure of which is incorporated herein by reference in its entirety. The International Application was published under PCT Article 21(2) in a language other than English. The applicant(s) herein explicitly rescind(s) and retract(s) any prior disclaimers or disavowals made in any parent, child or related prosecution history with regard to any subject matter supported by the present application.

### TECHNICAL FIELD

The present invention relates to a system for assisting in the improvement of sleep problems, wherein the system identifies the causes of a user's sleep problem by using a computer system, automatically generates a remedy for the sleep problem, and provides the user with the remedy.

### BACKGROUND ART

The results of numerous studies conducted by many researchers have shown that sleep problems are detrimental to a wide variety of physical and mental health, increase the risk of obesity, diabetes, and other similar diseases, increase the risk of cardiovascular diseases, and are also linked to cancer. Sleep problems also increase the risk of depression and other mental disorders, which in turn increases the risk of suicide and consequently increases mortality.

Furthermore, sleep problems not only damage physical and mental health, but also cause huge loss of money due to decreased productivity, and increase the risk of traffic accidents and serious work errors (for example, in the Chernobyl nuclear power plant accident and the Challenger explosion, sleep problems of the workers in charge were cited as one of the causes). According to the inventors' research, sleep problems have been shown to cause a decrease in productivity of approximately 3% in Japan, and this has been published as a paper (see Non-Patent Literature 1).

There are a very large number of people who have sleep problems; for example, one in two Japanese people is said to have some kind of sleeping problem, but there are very few doctors and medical professionals who specialize in sleep, and it is hard to say that there is a well-established social environment that allows those who have sleep problems to solve their problems.

Although many inventions have been proposed to automatically measure the sleep state using various mechanical devices and to determine sleep quality and other factors relating to sleep, there is no system that identifies the causes of sleep problems (especially erroneous lifestyle) and provides measures to remedy the causes. While sleep problems are extremely important for public health, the system to care for sleep problems is vulnerable and access to sleep medicine is difficult.

In addition, there is no system that automatically diagnoses and assesses sleep problems and sleep disorders and informs the person with sleep disturbance and his/her sleep instructor on how to address these issues. Furthermore, there is no system that has been scientifically proven to be effective.

Furthermore, there are only a limited number of known incorrect kinds of lifestyle that cause sleep problems, and it is not clear what kinds of lifestyle are associated with each type of sleep problem, such as difficulty falling asleep, awakening in the middle of sleep, excessive daytime sleepiness, and use or dependence on sleeping pills, etc., or how important these kinds of lifestyle are for sleep. In addition, there is no system that can determine daily-life habits which should be improved and to which priorities should be given, and present them to the person undergoing the sleep condition assessment.

### BACKGROUND ART LITERATURE

### Non-Patent Literature

Non-Patent Literature 1: Studies on Productivity and Sleep: ISHIBASHI, Yoshiki; SHIMURA, Akiyoshi, Association between work productivity and sleep health: a cross-sectional study in Japan. Sleep health, 2020, 6.3:270-276.
Non-Patent Literature 2: Journal of the American Heart Association, 2017, 6.5: e005479.
Non-Patent Literature 3: Sleep, 2010, 33.9:1159-1164.
Non-Patent Literature 4: Chronobiology international, 2018, 35:8: 1045-1053.
Non-Patent Literature 5: Journal of affective disorders 135. 1-3 (2011): 10-19.
Non-Patent Literature 6: BMC psychiatry 16.1 (2016): 375.
Non-Patent Literature 7: Psychosomatic research 64.4 (2008): 443-449.
Non-Patent Literature 8: Sleep 31.8 (2008): 1097-1101.
Non-Patent Literature 9: Sleep 31.5 (2008): 619-626
Non-Patent Literature 10: Sleep 29.8 (2006): 1009-1014
Non-Patent Literature 11: European Heart Journal 32.12 (2011): 1484-1492
Non-Patent Literature 12: Diabetes Care 33.2 (2010): 414-420
Non-Patent Literature 13: Current Biology 22.10 (2012): 939-943.
Non-Patent Literature 14: International Journal of Epidemiology 38.3 (2009): 848-854.
Non-Patent Literature 15: British Journal of Cancer 99 (2008): 1502-1505
Non-Patent Literature 16: British Journal of Cancer 99 (2008) 176-178
Non-Patent Literature 17: American journal of epidemiology 173.11 (2011): 1272-1279.
Non-Patent Literature 18: Occup Environ Med 69.8 (2012): 551-556.

### PATENT LITERATURE

Patent Literature 1: International Patent Laid-open No. WO2015/125477
Patent Literature 2 Japanese Patent Laid-open No. 2013-099507
Patent Literature 3 Japanese Patent Laid-open No. 2016-122347
Patent Literature 4 Japanese Patent Laid-open No. 2019-195469
Patent Literature 5 Japanese Patent Laid-open No. 2019-204444
Patent Literature 6 Japanese Patent Laid-open No. 2020-054839

### SUMMARY OF THE INVENTION

### Problems to be Solved by the Invention

An object of the present invention is to improve physical and mental health, prevent illnesses, remedy disorderedness, and improve the productivity and achieve other advantages in the workplaces/the scope of work by building a system that provides methods and solutions for determining sleep disorders and identifying the causes of sleep problems based on the relationship between the lifestyle and the sleep problems revealed by the inventors' research, and improving lifestyles that cause the sleep problems.

### Means for Solving the Problems

To achieve the object described above, the present invention relates to a system that identifies causes of a sleep problem of a user by using a computer system, automatically creates and provides remedy plans for the sleep problem, and provides the user and/or an administrator with the created remedy plans, the system including:
a user terminal and/or an administrator terminal; a server that operates the system; a database built in the server, a processing program that is downloaded to the server and operates the system; means for monitoring a sleep duration and sleep schedules; and means for monitoring sleep quality and a daytime function.

The processing program causes the server to perform each of the following processes: selecting candidate causes based on a type of the sleep problem; making a final determination of the causes of the sleep problem; and prioritizing and creating methods for addressing the finally determined causes.

The means for monitoring a sleep duration and sleep schedules is any of: means for displaying a questionnaire to the user on the user terminal and recording a result of a response to the questionnaire in a database A; a device that is worn by the user and measures a sleep state of the user, and means for recording a result of the measurement in the database A; a device that is attached to bedding and measures the sleep state of the user, and means for recording a result of the measurement in the database A; and a device that measures the sleep state of the user by means of a video system, and means for recording a result of the measurement in the database A.

The means for monitoring sleep quality and a daytime function is any of means for displaying a questionnaire to the user on the user terminal and recording a result of a response to the questionnaire in a database B; a device that is worn by the user and measures the sleep state of the user, and means for recording a result of the measurement in the database B; a device that is attached to the bedding and measures the sleep state of the user, and means for recording a result of the measurement in the database B; and a device that measures the sleep state of the user by means of a video system, or means for recording a result of the measurement in the database B.

The means for selecting candidate causes based on the type of the sleep problem is a means for identifying which type of the sleep state of the user is when the sleep state meets a predetermined criterion, based on information recorded in databases A and B and the predetermined criterion: a difficulty type in falling asleep, a difficulty type in maintaining sleep, or an excessive daytime sleepiness type; and provisionally identifying the causes of the problem based on a table that stores in advance probable causes of each of the difficult type in falling asleep, the difficulty type in maintaining sleep, and excessive daytime sleepiness type.

The means for making a final determination of the causes of the sleep problem is a means for making a final determination of whether the provisionally identified causes are true or not based on data on a result of a response to an additionally given questionnaire and predetermined criterion.

The means for prioritizing and creating countermeasures for the finally determined causes is a means for prioritizing contents of lifestyle to be improved, based on a lifestyle standardized coefficient table created in advance based on the causes of the problem that have been finally determined to be true and standardization coefficients of an impact of the lifestyle on the finally determined problem, in descending order of the standardization coefficients, and transmitting the contents to the user terminal and/or the administrator terminal, or outputting the contents in a form of a printed paper medium.

### Effects of the Invention

The present invention having the configuration described above can solve sleep problems that a user himself/herself has by identifying the causes of the user's sleep problems by using a computer system, presenting specific remedies for lifestyles based on the identified causes to the user, and encouraging the user to practice the remedies for improvement in mental and physical health, prevention of illnesses, alleviation of disorderedness, and improvement in the productivity in the workplaces/the scope of work, etc.

### BRIEF DESCRIPTION OF THE DRAWINGS

FIG. 1: Scope of causes of sleep problems identified by a system according to the present invention.

FIG. 2: Means A to E and the contents of processes carried out thereby.

FIG. 3: Types of the sleep problems and suspected causes (Table 1).

FIG. 4: Standardized coefficients of an impact of the lifestyle on the sleep problems (Table 2).

FIG. 5: Example of an implemented questionnaire.

FIG. 6: Example of an evaluation report to be submitted (output) to a user and/or an administrator.

FIG. 7: Results of a randomized controlled trial (RCT) given to students.

### MODE FOR CARRYING OUT THE INVENTION

The following is a detailed description of an embodiment of the present invention.

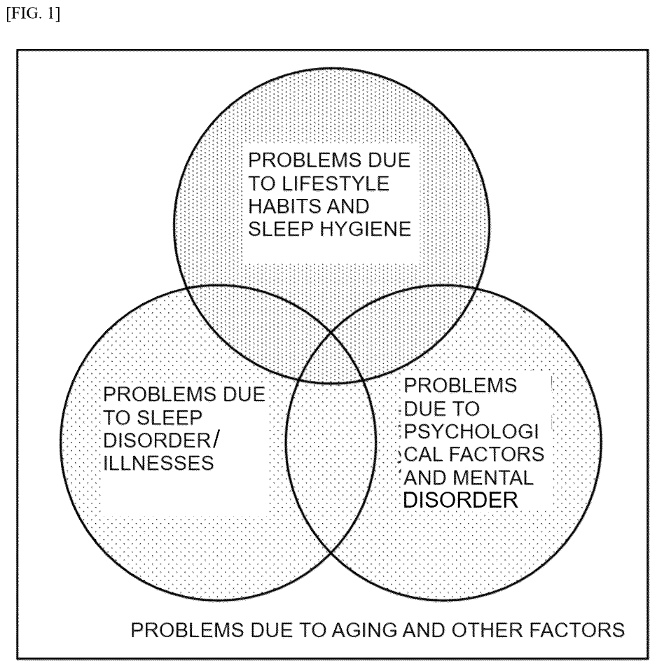

Sleep problems and causes thereof span a wide range, and each individual has various causes. As shown in FIG. 1, the scope of causes of sleep problems identified by a system according to the present embodiment includes “lifestyles and sleep hygiene”, “sleep disorders/illnesses”, and “psychological factors and mental disorders” excluding aging and other factors.

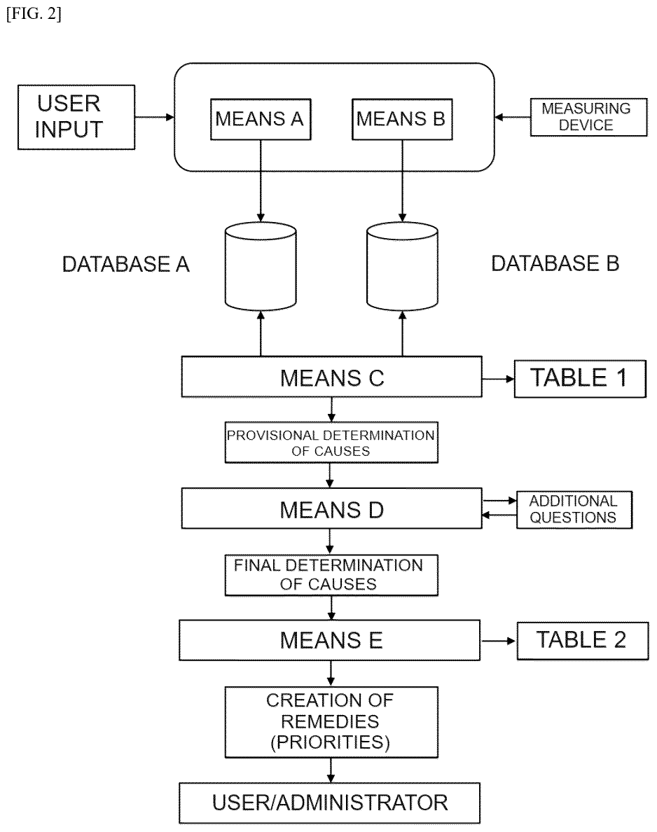

First, an overview of the present system is described. The system according to the present invention is a system that identifies the causes of a user's sleep problems by using a computer system, automatically creates remedies for the sleep problems, and provides the user and/or an administrator with the created remedies, and includes a user terminal and/or an administrator terminal, a management server that operates the system, databases (A and B) built in the management server, and a processing program that is downloaded to the management server and operates the entire system. The processing program executes five processes by using A: means for monitoring a sleep duration and sleep schedule, B: means for monitoring sleep quality and daytime functions, C: means for selecting candidate causes based on the type of sleep problem, D: means for making final determination (finalization and identification) of the causes of the sleep problem, and E: means for prioritizing and creating methods for addressing the finally determined causes. The means A to E and the contents of processes carried out thereby will be described below with reference to FIG. 2.

### A: Means for Monitoring Sleep Duration and Sleep Schedule

It is extremely important to know at what time the user goes to bed, how long it takes to fall asleep (sleep onset latency), and at what time the user wakes up in order to evaluate sleep problems and identify the causes thereof. To this end, the means A acquires data on bedtime, the sleep onset latency, wake-up time, and the sleep duration by using any of the following methods (A-1) to (A-4), and records the data in the database A.

### (A-1) Input Performed by User Via User Terminal

Using the user terminal, such as a PC or a smartphone, in which an application program of the present system is saved, the user starts the application program and inputs the date, the bedtime, the sleep onset time, the wake-up time, and the sleeping period, via a data input screen displayed on the user terminal. The user may instead access a website that operates the present system via the user terminal or any other device, and input the items described above via a data input screen that appears on the website. The input date and points of time are recorded in the database A. The information is not necessarily input on the same day, but may be input on a later date. The sleep duration may be automatically calculated by the system based on the falling-asleep time and the wake-up time.

The system automatically calculates an average value of each data point whenever the data is input daily, and makes determination that will be described later based on the average value. Note that depending on the type of determination, the system refers to the actual time instead of the average value because variations in the time at which the use went to bed, the sleep onset time, and the time at which the user woke up are observes (the same applies to the construction of data measured by other methods described below).

When response data is available from an existing sleep questionnaire, such as the Pittsburgh Sleep Quality Index (PSQI) or Munich Chronotype Questionnaire (MCTQ), or a questionnaire or a sleep diary in which the sleep period of the day is recorded by the user or his/her guardian/healthcare provider, the data may be recorded in the database A.

Pittsburgh Sleep Quality Index (PSQI): Buysse, Daniel J., et al. “The Pittsburgh Sleep Quality Index: a new instrument for psychiatric practice and research.” Psychiatry Research 28.2 (1989): 193-213.

Munich Chronotype Questionnaire (MCTQ): Roenneberg, Till, Anna Wirz-Justice, and Martha Merrow. “Life between clocks: daily temporal patterns of human chronotypes.” Journal of biological rhythms 18.1 (2003): 80-90.

Each of the data described above may be read by an OCR or other means from responses made by the user and written on paper, and may be saved in the database A.

Furthermore, each of the data described above may be input from another system or application program connected to the present system via a network through API (Application Programming Interface) connection.

### (A-2) Measurement Made by Device Worn on Human Body

The bedtime, the sleep latency, sleep onset time, the wake-up time, and the sleep duration are automatically measured by a device worn on the human body, and the measured data is recorded in the database A.

For example, one or more of the following devices: polysomnography (PSG); electroencephalography; optical topography; electrocardiogra pulsation measurement; magnetoencephalography (EMG); skin potential measurement; and deep body temperature measurement are used to detect appearance of an electroencephalograph or an magneto-encephalograph characteristic of sleep, changes in cerebral blood flow rate, decreases in pulse rate and increases in parasympathetic activity, decreases in sympathetic activity to parasympathetic activity (LF/HF ratio), decreases in activity (acceleration), decreases in deep body temperature, and other factors to automatically measure the time at which the user went to bed, sleep onset latency, the time at which the user would have fallen asleep, the time at which the user woke up, and the sleep duration. Each of the measured data is transmitted to the present system using a communication device and recorded in the database A built in the server. The data measured by each of the measuring devices may be recorded in the database A via a recording medium such as a USB memory without going through the communication device (the same applies hereinafter).

### (A-3) Measurement Made by Devices Attached to Bedding

A pressure-sensitive sensor, an acceleration sensor, a vibration sensor, a magnetic sensor, a temperature sensor, a voltage sensor, or the like is placed under the bedding or on the bedding near the user, and the time at which the user went to bed, sleep onset latency, the time at which the user would have fallen asleep, the time at which the user woke up, and the sleep duration are automatically measured based on decreases or fluctuations in locomotion, drops in body temperature, and other factors characteristic of sleep. Each of the measured data is transmitted to the present system using a communication device and recorded in the database A.

### (A-4) Measurement Made by Video Monitoring System

A video camera, an infrared camera, a motion detector, a thermographic device, or any other device that can monitor the sleep state is installed in the bedroom to automatically measure the bedtime, sleep latency, the sleep onset time, the wake-up time, and the sleep duration based on the decreases or fluctuations in locomotion, decreases in eye movement, drops in body temperature, and other factors characteristic of sleep. Each of the measured data is transmitted to the present system using a communication device and recorded in the database A.

### B: Means for Monitoring Sleep Quality and Daytime Functions

Assessing the presence or absence and severity of impairment of the sleep maintaining function, as typified by arousal/awakening during sleep, is extremely important in evaluating sleep problems and identifying causes thereof. In addition, the diagnosis of insomnia requires not only the fact that sleep problems have occurred but also the fact that problems with the daytime functions such as daytime sleepiness have occurred as a result of the sleep problems. Therefore, in this means, the quality of sleep and the presence or absence of daytime sleepiness are measured by any of the following methods (B-1) to (B-4), and the results of the measurement are recorded in the database B.

### (B-1) Input Performed by User Via User Terminal

Using the user terminal, such as a PC or a smartphone, in which an application program of the present system is saved, the user starts the application program and inputs the date, whether or not the user has awakened in the middle of sleep, the number of times and length of the mid-sleep awakenings, how easy it is for the user to fall asleep again if the user awakes in the middle of sleep, and the number of times the user has dreamed and the subjective lengths thereof, via a data input screen displayed on the user terminal. The user also inputs the degree of daytime sleepiness and the frequency of napping or dozing. The user may instead access a website that operates the present system via the user terminal or any other device, and input the items described above via a data input screen that appears on the website. The input contents are recorded in the database B. The information is not necessarily input on the same day, but may be input on a later date. When the user takes any sleeping pills or other sleep-related drugs, such as sedative drugs, the user may also input information on the drug, but this is not a particularly essential requirement and is therefore omitted in the present system.

The system automatically calculates an average value of each of the data whenever the data is input daily, and makes determination that will be described later based on the average value.

When there is data on responses to an existing sleep questionnaire, such as the Pittsburgh Sleep Quality Index (PSQI) and/or the Athens Insomnia Scale (AIS) and/or the Insomnia Severity Scale (ISI) and/or a questionnaire in which the user responds to questions about any mid-sleep awaking or the sleep quality, the questionnaire data may be recorded in the database B. Note that these existing questionnaires also include survey items on staying asleep disorders indicating the sleep quality, daytime sleepiness, and presence or absence of dysfunction, and use of sleeping pills.

Athens Insomnia Scale (AIS): Soldatos, Constantin R., Dimitris G. Dikeos, and Thomas J. Paparrigopoulos. “Athens Insomnia Scale: validation of an instrument based on ICD-10 criteria.” Journal of psychosomatic research 48.6 (2000): 555-560.

Insomnia Severity Scale (ISI): Bastien, Celyne H., Annie Vallieres, and Charles M. Morin. “Validation of the Insomnia Severity Index as an outcome measure for insomnia research.” Sleep medicine 2.4 (2001): 297-307.

The data described above may be read by an OCR or other means from responses made by the user and written on paper, and may be recorded in the database B. Each of the data described above may instead be input on behalf of the user by a third party such as a physician or a nurse who is aware of the user's situation.

The data described above may still instead be input via another system or application connected to the present system via a network through an API (Application Programming Interface) connection.

### (B-2) Measurement Made by Device Worn on Human Body

Sleep quality is automatically measured by using devices worn on the human body, such as polysomnography (PSG) for whole-night sleep, electroencephalography (EEG), optical topography, electrocardiogra pulsation measurement, magnetoencephalography (EMG), and skin potential measurement. The device measures the periods during which appearance of the brain waves of the user who is awake, changes in cerebral blood flow, transient increases in pulse rate, and transient increases in sympathetic nerve activity characteristic of mid-sleep awakening or extremely light sleep are observed. Each of the measured data is transmitted to the present system using a communication device and recorded in the database B.

The presence or absence of daytime sleepiness or napping are automatically measured as sleepiness or napping based on the appearance of LVMF brain waves, increases in delta power, temporary increases in parasympathetic activity, or marked decreases in eye movement, which are characteristic of strong sleepiness, by using an electroencephalograph, electrocardiogram measurement, skin potential measurement, or any other measurement. Each of the measured data is transmitted to the present system using a communication device and recorded in the database B.

### (B-3) Measurement Made by Device Installed in Bedding

A pressure-sensitive sensor, an acceleration sensor, a vibration sensor, a magnetic sensor, a temperature sensor, or any other sensor is placed under the bedding or on the bedding near the user, and the time when a characteristic increase in accelerated activity, responses of the user who is awake, or transient increases in body temperature are observed at the time when the user awakes in the middle of sleep or when sleep becomes significantly shallow is determined as the mid-sleep awakening. The frequency and duration of the awakening are automatically measured. Each of the measured data is transmitted to the present system using a communication device and recorded in the database B.

### (B-4) Measurement Made by Video Monitoring System

A camcorder, an infrared camera, a motion detector, a thermographic device, or any other device that can monitor the sleep state is installed in the bedroom, and the time when a characteristic increase in locomotion or transient increases in body temperature are observed at the time when the user awakes in the middle of sleep or when sleep becomes significantly shallow is determined as the mid-sleep awakening. The frequency and duration of the awakening are automatically measured. Each of the measured data is transmitted to the present system using a communication device and recorded in the database B.

### C: Means for Presenting Candidate Causes Based on Type of Sleep Problem

The means for presenting candidate causes based on the type of the sleep problem is means for inferring from the data obtained by each of the means A and B described above whether or not there is a problem with the user's sleep, and if so, what the cause of the problem is. Research conducted by the inventors has shown that most sleep problems are caused by one or more of the reasons listed below. Furthermore, the inventors' research has also shown that each type of sleep problem has different potential causes, so that the present means is configured to automatically extract potential candidate causes for each type of sleep problem. There are three major types of sleep problems, “difficulty falling asleep”, “difficulty maintaining sleep”, and “excessive daytime sleepiness”. The system first identifies which type of sleep problem the user has, “difficulty falling asleep”, “difficulty maintaining sleep”, or “excessive daytime sleepiness” from the information recorded in the databases A and B described above. For example, when a sleep problem meets a predefined criteria of “30 minutes or more is required from bedtime to sleep onset,” the sleep problem is determined to be of the type “difficulty falling asleep,” when the user “awakens three or more times”, the sleep problem is determined to be of the type “difficulty maintaining sleep”, and when the user takes “daytime a nap three or more times a week”, the sleep problem is determined to be of the type “excessive daytime sleepiness”.

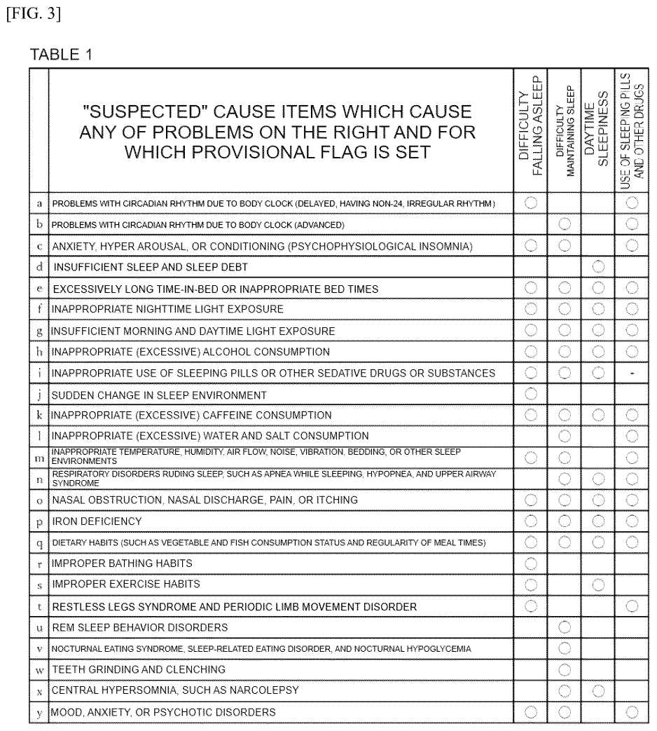

According to the inventors' research, it is clear that the problems of “difficulty falling asleep”, “difficulty maintaining sleep”, and “excessive daytime sleepiness” are closely related to causes a to y, as indicated by Table 1 shown in FIG. 3.

After determining which of the above three types of sleep problems the user has, the system provisionally identifies which of the candidate causes from a to y is strongly suspected by using Table 1 shown in FIG. 3. Any one of the contents a to y listed below, each having multiple problems or causes, is not determined as only one cause or candidate cause. The details of each of the sleep problems and how to further narrow down these candidates will be described later. The “use of sleeping pills and other sedative drugs”, “circadian rhythm problems due to body clock”, and “problems caused by mood, anxiety, and psychotic disorders” among the contents shown in Table 1 are known to the inventors, who have published research on these issues in the past. However, the inventors only studied these associations by themselves, which detract none from the novelty of the present invention, namely, “investigation of multiple causes” and “weighting of the causes”.

Shimura, Akiyoshi, et al. “Later sleep schedule and depressive symptoms are associated with usage of multiple kinds of hypnotics.” Sleep medicine 25 (2016): 56-62.

The inventors have also published previous studies on the relationship between the total PSQI score, which vaguely describes overall sleep problems, and the age, gender, marital status, residential situation, overtime hours, commuting time, occupational stress, dietary regularity, breakfast consumption situation, dinner consumption situation, vegetable consumption situation, frequency of nighttime drinking, weight fluctuations, light environment, and caffeine consumption. However, the previous studies do not clarify specific sleep problems such as difficulty falling asleep or difficulty maintaining sleep, or the risk of each symptom, and thus do not undermine the novelty of the present invention, as will be described later.

Shimura, Akiyoshi, et al. “Which sleep hygiene factors are important? Comprehensive assessment of lifestyle habits and job environment on sleep among office workers.” Sleep health 6.3 (2020): 288-298.

Candidate causes of sleep problems are listed below (see FIG. 3)
a. Those due to problems with the circadian rhythm due to the body clock (delayed, non-24, irregular rhythm)
b. Those due to problems with the circadian rhythm due to the body clock (advanced)
c. Those due to anxiety, hyperarousal, or conditioning (psychophysiological insomnia)
d. Those due to insufficient sleep/sleep debt
e. Those due to excessively long on-bed periods or inappropriate bedtimes
f. Those due to inappropriate nighttime light exposure
g. Those due to insufficient morning and daytime light exposure
h. Those due to inappropriate (excessive) alcohol consumption
i. Those due to inappropriate use of sleeping pills or other sedative drugs or substances
j. Those due to sudden change in sleep environment
k. Those due to inappropriate (excessive) caffeine consumption
l. Those due to inappropriate (excessive) water or salt consumption
m. Those due to inappropriate temperature, humidity, air flow, noise, vibration, bedding, or other sleep environments
n. Those due to respiratory disorders during sleep, such as apnea while sleeping, hypopnea, and upper airway syndrome
o. Those due to nasal obstruction, nasal discharge, pain, or itching
p. Those due to iron deficiency
q. Those due to dietary habits (such as vegetable and fish consumption status and regularity of meal times)
r. Those due to improper bathing habits
s. Those due to inappropriate exercise habits
t. Those due to restless legs syndrome/periodic limb movement disorder
u. Those due to REM sleep behavior disorder
v. Those due to nocturnal eating syndrome, sleep-related eating disorder, or nocturnal hypoglycemia
w. Those due to teeth grinding or clenching
x. Those due to central hypersomnia such as narcolepsy
y. Those due to mood, anxiety, or psychotic disorder

When sleep problem(s) is detected, the present means automatically determine whether or not the problem corresponds to any of the causes described above, or whether or not there is a possibility (suspicion) corresponding thereto in accordance with each of the patterns shown below, and extracts the determined causes or possibilities as candidates.

### <When User has Difficulty Falling Asleep>

When the system determines that the user has difficulty falling asleep based on the data recorded in the database A (for example, when the sleep onset latency from bedtime to falling asleep is 30 minutes or longer, or when the user himself/herself has responded that he/she has “trouble falling asleep”), the system extracts from the causes described above “problems with the circadian rhythm due to the body clock (delayed, non-24, irregular sleep-wake rhythm)”, “anxiety, hyperarousal, or conditioning”, “excessively long time-in-bed or inappropriate bedtimes”, “inappropriate nighttime light exposure”, “insufficient morning and daytime light exposure”, “inappropriate alcohol consumption”, “inappropriate use of sleeping pills or other sedative drugs or substances”, “sudden change in sleep environment”, “inappropriate caffeine consumption”, “inappropriate temperature, humidity, air flow, noise, vibration, bedding, or other sleep environments”, “nasal obstruction, nasal discharge, pain, or itching”; “iron deficiency”; “dietary habits (such as vegetable and fish consumption status and regularity of meal times)”, “inappropriate bathing habits”, “inappropriate exercise habits”, “restless legs syndrome/periodic limb movement disorder”, and “mood, anxiety, or psychotic disorder” as highly suspicious causes, then provisionally flag the extracted causes, and identify the final problem by the method described later.

<when User has Difficulty Maintaining Sleep>

When the system determines that the user has the problem of difficulty maintaining sleep based on the data recorded in the database A (for example, when frequent arousal responses are observed during sleep, or the proportion of deep sleep decreases, when the user awakens within a short period after falling asleep, or when the user reports frequent awakenings during the night, awakens early in the morning, or have nightmares), the system extracts from the causes described above “problems with the circadian rhythm due to the body clock (advanced phase)”, “anxiety, hyperarousal, or conditioning (psychophysiological insomnia)”, “excessively long time-in-bed or inappropriate bedtimes”, “inappropriate nighttime light exposure”, “insufficient morning and daytime light exposure”, “inappropriate alcohol consumption”, “inappropriate use of sleeping pills or other sedating drugs or substances”, “inappropriate caffeine consumption”, “inappropriate water and salt consumption”, “inappropriate temperature, humidity, air flow, noise, vibration, bedding, or other sleep environments”, “respiratory disorders during sleep, such as apnea while sleeping, hypopnea, and upper airway syndrome”, “nasal obstruction, nasal discharge, pain, or itching”, “iron deficiency”, “dietary habits (such as vegetable and fish consumption status and regularity of meal times)”, “inappropriate exercise habits”, “restless legs syndrome/periodic limb movement disorder”, “REM sleep behavior disorder”, “nocturnal eating syndrome, sleep-related eating disorder, or nocturnal hypoglycemia”, “teeth grinding or clenching”, “central hypersomnia such as narcolepsy”, and “mood, anxiety, or psychotic disorder” as highly suspicious causes, then provisionally flag the extracted causes, and identify the final problem by the method described later.

<when User Suffers from Excessive Daytime Sleepiness>

When the system determines that the user has the problem of excessive daytime sleepiness based on the data recorded in the database B (for example, when the device detects during the user's active hours appearance of an electroencephalograph/magnetoencephalography characteristic of sleep, changes in cerebral blood flow, decreases in pulse rate or increases in parasympathetic activity, decreases in LF/HF ratio, or decreases in amount of activity (acceleration), or when the user himself/herself responds that the user frequently suffers from excessive daytime sleepiness or sleep attacks), the system extracts “lack of sleep or sleep debt”, “excessively long on-bed periods or inappropriate bedtimes”, “inappropriate nighttime light exposure”, “insufficient morning and daytime light exposure”, “inappropriate alcohol consumption”, “inappropriate use of sleeping pills or other sedating drugs or substances”, “inappropriate caffeine consumption”, “sleep apnea, hypopnea, upper airway syndrome, or other breathing problems during sleep”, “respiratory disorders during sleep, such as apnea while sleeping, hypopnea, and upper airway syndrome”, “nasal obstruction, nasal discharge, pain, or itching”, “iron deficiency,” “dietary habits (such as vegetable and fish consumption status and regularity of meal times)”, “inappropriate exercise habits”, and “central hypersomnia such as narcolepsy” as highly suspicious causes, then provisionally flag the extracted causes, and identify the final problem by the method described later.

<when User Uses Sleeping Pills or Other Sedative Drugs/Substances>

As mentioned above, using or taking sleeping pills or other drugs is not an essential requirement of the present invention, but can be attributed to “problems with the circadian rhythm due to the body clock (delayed, non-24, irregular rhythm)”, “problems with the circadian rhythm due to the body clock (advanced)”, “advanced sleep phase syndrome”, “anxiety, hyperarousal, or conditioning (psychophysiological insomnia)”, “excessively long on-bed periods or inappropriate bedtimes”, “inappropriate nighttime light exposure”, “insufficient morning and daytime light exposure”, “inappropriate alcohol consumption”, “inappropriate caffeine consumption”, “inappropriate water and salt consumption”, “inappropriate temperature, humidity, air flow, noise, vibration, bedding, or other sleep environments”, “respiratory disorders during sleep, such as apnea while sleeping, hypopnea, and upper airway syndrome”, “nasal obstruction, nasal discharge, pain, or itching,” “iron deficiency”, “dietary habits (such as vegetable and fish consumption status and regularity of meal times)”, “restless legs syndrome/periodic limb movement disorder”, and “mood, anxiety, or psychotic disorder” as highly suspicious causes, which have been revealed by the inventors' research, and may be incorporated into the present system.

### D: Means for Final Determination (Finalization or Identification) of Candidate Causes of Sleep Problems

With regard to the causes of the sleep problems inferred by the means C described above, whether or not each of the selected candidate causes is true is determined by the following method based, for example, on the data recorded in the databases A and B, data from responses to additional questions to the user, and various existing data pertaining to the user, and the true cause is finally narrowed down, finalized, and identified.

<a. Problems with the Circadian Rhythm Due to the Body Clock (Delayed, Non-24, Irregular Rhythm)>

When the user frequently wakes up at delayed times, and the sleep onset latency is long when the user goes to bed early, or when there is a noticeable delay in MSFsc time, which is a result of a known sleep rhythm questionnaire, or the MEQ score, which is another result of the known sleep rhythm questionnaire, is low, the system makes a provisional determination (sets provisional flag) that “there is a suspicion that the sleep problem is caused by problems with the circadian rhythm due to the body clock (delayed, non-24, irregular rhythm)”. To check whether the provisional determination is correct or not, the system automatically generates the following questions and prompts the user to respond to them, or displays from the beginning the questions on the user terminal and prompts the user to respond to them. When the system receives more positive responses to the following question items, the system makes a final determination (sets a finalization flag) that “there is a stronger suspicion that the sleep problem is caused by problems with the circadian rhythm due to the body clock (delayed, non-24, irregular rhythm)”. The provisional determination and final determination hereinafter mean that the system sets a provisional flag and a finalization flag, respectively. The final determination further means finalization and identification.
Whether it is not easy but difficult to wake up early in the morning.
Out of the morning time zone or the afternoon time zone, whether the user feels better in the afternoon time zone.
When the user is woken up or the alarm goes off early in the morning, whether the user sometimes has no memory of the fact described above, or whether or not are the user's characters sometimes altered.
Whether the user has trouble sleeping for a while after going to bed early in the evening.
Whether difficulty falling asleep is reduced or eliminated if the user goes to bed late at night or early in the morning.
Whether the user sometimes has sleepiness in the evening instead of in the afternoon.
Whether the user has difficulty eating breakfast, or whether having breakfast makes the user feel ill.
Whether the user feels hungry in the middle of the night.

The following data may be acquired from measuring devices and used in place of the questions described above or used as a further aid in the final determination. According to the inventors' research, when a larger number of characteristics described below are acquired, it is clear that “there is a stronger suspicion that the sleep problem is caused by problems with the circadian rhythm due to the body clock (delayed, non-24, irregular rhythm)”.
Delay in the center time in the sleep period
Total illuminance measured over the period from sunrise to noon
Total illuminance measured over the period from sunset to sunrise
Delay in center-of-gravity time of data on total amount of activity
Delay in peak time of autonomic nervous activity or delay in center-of-gravity time of total amount of autonomic nervous activity
Delay in the time of the lowest point of deep body temperature
   <b. Problems with the Circadian Rhythm Due to the Body Clock (Advanced)>

When the user frequently experiences an advance of the overall sleep period and associated early morning awakenings, or when there is a noticeable advance in MSFsc time, which is a result of a known sleep rhythm questionnaire, or the MEQ score, which is another result of the known sleep rhythm questionnaire, is high, the system makes a provisional determination that “there is a suspicion that the sleep problem is caused by problems with the circadian rhythm due to the body clock (advanced)”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by problems with the circadian rhythm due to the body clock (advanced)”.
Whether the user tends to be sleepy in the early evening or evening.
Whether the user is able to sleep when going to bed in the early evening or evening.
Whether the user wakes up early in the morning or before dawn without an alarm.

The following data may be acquired from measuring devices or other devices and used in place of the questions described above or used as a further aid in the final determination. According to the inventors' research, when a larger number of characteristics described below are acquired, it is clear that “there is a stronger suspicion that the sleep problem is caused by problems with the circadian rhythm due to the body clock (advanced)”.
Older ages
Advance of the midpoint of the sleep period
Total illuminance measured over the period from sunrise to noon
Total illuminance measured over the period from sunset to sunrise
Advance of center-of-gravity time of data on total amount of activity
Advance of peak time of autonomic nervous activity or advanced of center-of-gravity time of total amount of autonomic nervous activity
Advance of the time of the lowest point of deep body temperature
   <c. Anxiety, Hyperarousal, or Conditioning (Psychophysiological) Insomnia>

When the user has difficulty falling asleep or staying asleep and is suspected of being hyper-aroused, the system makes a provisional determination that “there is a suspicion that the sleep problem is caused by anxiety, hyperarousal, or conditioning”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by anxiety, hyperarousal, or conditioning”.
Whether the user is anxious about being able to sleep at night
Whether the user frequently becomes wakeful when going to bed despite feeling sleepy
Whether the user frequently falls asleep while thinking about what to do tomorrow
Whether the user frequently goes to bed without feeling sleepy
Whether the user goes to bed early because of fear of not being able to sleep
Whether the user frequently engages in activities other than sleeping in bed
Whether the user easily falls asleep at places other than the usual sleeping place, such as a travel destination

The following data may be acquired from measuring devices or other devices and used in place of the questions described above or used as a further aid in the final determination. According to the inventors' research, when a larger number of characteristics described below are acquired, it is clear that “there is a stronger suspicion that the sleep problem is caused by anxiety, hyperarousal, or conditioning”.
A lack of decrease or increase in the ratio of sympathetic to parasympathetic activity (LF/HF ratio) after the user goes to bed
Magnitude of activity upon going to bed or detection of any movements
   <d. Insufficient Sleep/Sleep Debt>

When the user suffers from excessive daytime sleepiness, or when it is believed that the user has only been asleep for a period shorter than required, the system makes a provisional determination that “there is a suspicion that the sleep problem is caused by insufficient sleep/sleep debt”. To check whether the provisional determination is correct or not, the system compares the user's actual sleep duration with an average sleep duration derivable from the user's age based on the information produced by measuring devices and recorded in the database A or responses to a questionnaire (Reference: Ohayon, Maurice M., et al. “Meta-analysis of quantitative sleep parameters from childhood to old age in healthy individuals: developing normative sleep values across the human lifespan.” Sleep 27.7 (2004): 1255-1273).

As a result of the comparison, when the user's sleep duration is shorter than the average value and/or the sleep onset latency is significantly short and/or the sleep efficiency (total sleep duration divided by total on-bed period) is very high and close to one, the system makes a final determination that “there is a strong suspicion that the sleep problem is caused by insufficient sleep/sleep debt”. At this point of time, the system may additionally ask the user how much sleep the user thinks is desirable to see if the user underestimates the required sleep duration, or if the user has a false belief that humans are fine without sleep.

<e. Excessively Long On-Bed Periods or Inappropriate Bedtimes>

When the user has difficulty falling asleep or difficulty maintaining sleep, suffers from excessive daytime sleepiness, or uses sleeping pills or other sedating drugs or substances and stays in bed for a period longer than a required sleep duration, or goes to bed or tries to fall asleep at inappropriate times, the system makes a provisional determination that “there is a suspicion that the sleep problem is caused by excessively long on-bed periods or inappropriate bedtimes. To check whether the provisional determination is correct or not, the system compares the user's actual sleep duration with an average sleep duration derivable from the user's age based on the information produced by measuring devices and recorded in the database A, a database containing a record of the sleep period, or responses to a questionnaire. When the user stays in bed for a period longer than the average value and sleeps with poor efficiency (total sleep duration divided by total on-bed period), or continues to stay in bed after the user wakes up, the system makes a final determination that “there is a strong suspicion that the sleep problem is caused by excessively long on-bed periods”.

In addition, as known studies have shown, due to the effects of the circadian rhythm (body clock), the period from a habitual wake-up time or times therearound to about five hours thereafter, and about two to four hours before a habitual falling-asleep time are “sleep forbidden zones,” which are time zones during which it is difficult for the user to fall asleep. Therefore, when the user sleeps in these time zones, the system determines that the user is trying to fall asleep at an inappropriate time. When the user works in shifts and labor management data or the user's responses show that the user tries to fall asleep immediately in the morning after the night shift ends at a habitual wake-up time or times therearound or times within about five hours thereafter, or when the user tries to fall asleep two to four hours before the time the user normally falls asleep before the night shift, the system further flags representing that “there is a suspicion that the sleep problem is caused by shift work or the user suffers from a shift work sleep disorder”.

References: Lavie, P. (1986). Ultrashort sleep-waking schedule. III. ‘Gates’ and ‘forbidden zones’ for sleep. Electroencephalography and clinical neurophysiology, 63, 414-425, Existing technology: U.S. Pat. No. 6,722,911

<f. Inappropriate Nighttime Light Exposure>

When the user has difficulty falling asleep or staying asleep, or suffers from excessive daytime sleepiness, the system makes a provisional determination that “there is a suspicion that the sleep problem is caused by inappropriate nighttime light exposure”. To check whether the provisional determination is correct or not, the system additionally investigates whether excessive light exposure occurs during the night after sunset or during sleep. When a measurement device worn on the user or an illuminance meter installed in a room continuously detects an illuminance approximately ranging from 30 to 50 lx or higher at night, a blue wavelength illuminance of 10 lx or higher, or an illuminance of 3 lx or higher while the user is sleeping, the system makes a final determination that “there is a strong suspicion that the sleep problem is caused by inappropriate nighttime light exposure”.

The system automatically generates the following questions and prompts the user to respond to them, or displays from the beginning the questions on the user terminal and prompts the user to respond to them. When the system receives more positive responses to the following questions, the system determines that there is a stronger suspicion that the sleep problem is caused by inappropriate nighttime light exposure.
Whether the user spends a long time at night in a brightly lit environment such as an office or not.
Whether the nighttime lighting is white or not.
Whether the nighttime lighting is indirect or not.
Whether the user frequently or for long periods of time uses electronic displays such as a TV, PC, or smartphone after sunset or not.
Whether the user uses electronic displays such as a handheld game console or smartphone in bed or not.
Whether the user is exposed to light during sleep or not (whether the user sometimes falls asleep without turning off lights, or whether light enters the room through a window or door, causing the brightness to generally exceed 3 lx or not).
   <g. Insufficient Morning and Daytime Light Exposure>

When the user has difficulty falling asleep or staying asleep, or suffers from excessive daytime sleepiness, the system makes a provisional determination that “there is a suspicion that the sleep problem is caused by insufficient morning and daytime light exposure”. To check whether the provisional determination is correct or not, the system additionally investigates whether light exposure is in sufficient during the period around the wake-up time or during daytime activities. When a measuring device worn on the user or an illuminance meter installed in the room where the user spends his/her time does not detect an illuminance ranging from about 50 to 300 lx or higher around the user's wake-up time or the sunrise, or when the user spends his/her time in multiple rooms and the total period over which the measuring device worn on the user or multiple illuminance meters installed in the multiple rooms detect a total illuminance ranging from about 500 to 1000 lx or higher for each period over which the user stays in the rooms from the sunrise to the sunset is shorter than one hour, the system makes a final determination that “there is a strong suspicion that the sleep problem is caused by insufficient morning and daytime light exposure”

The system automatically generates the following questions and prompts the user to respond to them, or displays from the beginning the questions on the user terminal and prompts the user to respond to them. When the system receives more positive responses to the following questions, the system determines that the above suspicion is stronger.
The rooms are not exposed to morning sunlight due to use of light blocking curtains, storm shutters, or other objects.
The rooms are dark at the wake-up time or in the morning because windows of the rooms face west or the rooms do not get a lot of sunlight.
For example, the user stays indoors, works indoors most of the time, or is not exposed to outdoor light when commuting to and from work, so that the user spends less than one hour a day in outdoor light or an equivalent high-illuminance environment (1000 lx or higher).
   <h. Inappropriate (Excessive) Alcohol Consumption>

When the user has difficulty falling asleep or staying asleep, suffers from excessive daytime sleepiness, or uses sleeping pills or other sedating drugs or substances, the system makes a provisional determination that “there is a suspicion that the sleep problem is caused by inappropriate alcohol consumption”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by inappropriate alcohol consumption”.
Whether the user frequently consumes alcohol within four to five hours before bedtime in amounts sufficient to cause intoxication, sleepiness, or some other psychoactive effect.
Whether the user has a nightcap.
Whether the user frequently consumes alcohol in the daytime.
Whether the user drinks alcohol every day or at a frequency close thereto.
Whether the user experiences irritability or mental/physical discomfort when not consuming alcohol.
Whether the user has difficulty resisting the desire to consume alcohol.
Whether the user has ever been diagnosed as an alcoholic or suspected of being an alcoholic.
Whether the user has responded positively to screening for alcohol dependence as typified by CAGE or AUDIT.
   <i. Inappropriate (Excessive) Use of Sleeping Pills or Other Sedating Drugs or Substances>

When the user has difficulty falling asleep or staying asleep, or suffers from excessive daytime sleepiness, the system makes a provisional determination that “there is a suspicion that the sleep problem is caused by inappropriate use of sleeping pills or other sedating drugs or substances”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by inappropriate use of sleeping pills or other sedating drugs or substances”.
Whether or not does the user frequently use over-the-counter drugs or supplements claiming hypnotic effects?
Whether or not have the user been using any GABA receptor agonists or antihistamines readily transmitted to the central nervous system over a long period?
Whether or not does the user use short-acting GABA receptor agonists at night?
Whether or not does the user use high-titer GABA receptor agonists or antihistamines readily transmitted to the central nervous system at night?
Whether or not does the user use multiple (many) GABA receptor agonists or antihistamines readily transmitted to the central nervous system at night?
Whether or not does the user take any other sedative or somnolent drugs or substance dependence on which or tolerance for which has not been ruled out?
Whether or not does the user suffer from insomnia at a high rate when the user stops taking the substances or medications described above?
Whether or not does the user use alcohol and any of the substances or drugs described above at the same time?
   <j. Sudden Change in Sleep Environment>

When the user has difficulty falling asleep and there is a suspicion that the difficulty is caused by environmental hyperarousal or loss of conditioning or routine for falling asleep, the system makes a provisional determination that “there is a suspicion that the sleep problem is caused by a sudden change in sleep environment”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by a sudden change in sleep environment”.
Whether or not has the user moved?
Whether or not has the user changed his/her bedroom?
Whether or not has the user redecorated the bedroom?
Whether or not has the user changed his/her bedding?
Whether or not has the user found a bed partner?
Whether or not has the environment related to sleep changed for some other reason?
   <k. Inappropriate (Excessive) Caffeine Consumption>

When the user has difficulty falling asleep or staying asleep, suffers from excessive daytime sleepiness, or uses sleeping pills or other sedating drugs or substances, the system makes a provisional determination that “there is a suspicion that the sleep problem is caused by inappropriate caffeine consumption”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by inappropriate caffeine consumption”.
Whether the user frequently consumes caffeine-containing beverages after sunset or not.
Whether the user consumes caffeinated beverages such as coffee, nutritional drinks, energy drinks, tea, caffeinated soft drinks, or caffeinated foods at bedtime in an amount and timing likely to leave 50 mg of caffeine or more (with a half-life of three to seven hours) or not.
Whether the total daily caffeine consumption exceeds 400 mg (equivalent to approximately 400 to 600 ml of coffee or more, or 2 l of tea or more) or not.
Whether the user frequently experiences nightmares or not.
Whether the user frequently grinds or clenches his/her teeth or not.

When there is a database that the user himself/herself has recorded the time at which the user consumed caffeinated beverages and the amount thereof, the system may refer to the database, calculate an estimated amount of caffeine remaining in the user's body at the bedtime based on the caffeine half-life of three to seven hours, and make a final determination that “there is a strong suspicion that the sleep problem is caused by inappropriate caffeine consumption” based on whether the amount of remaining caffeine is 50 mg or greater or not.

<l. Inappropriate (Excessive) Water and Salt Consumption>

When the user has difficulty maintaining sleep or uses sleeping pills or other sedating drugs or substances, the system makes a provisional determination that “there is a suspicion that the sleep problem is caused by inappropriate (excessive) water and salt consumption”. To check whether the provisional determination is correct or not, the system automatically generates the following questions and prompts the user to respond to them, or displays from the beginning the questions on the user terminal and prompts the user to respond to them. As the system receives more positive responses to the following questions, the system makes a final determination that “there is a stronger suspicion that the inappropriate (excessive) water and salt consumption”.
Whether the user consumes only a small amount of water in the first half of the day or not.
Whether the user tends to consume a large amount of water during dinner, after dinner, and before going to bed or not.
Whether the total daily salt consumption is large or not.
Whether the amount of salt consumption is large at dinner or not.
   <m. Inappropriate Temperature, Humidity, Air Flow, Noise, Vibration, Bedding, or Other Sleep Environments>

When the user has difficulty falling asleep or staying asleep or uses sleeping pills or other sedating drugs or substances, the system makes a provisional determination that “there is a suspicion that the sleep problem is caused by inappropriate temperature, humidity, air flow, noise, vibration, bedding, or other sleep environments”. To check whether the provisional determination is correct or not, the system doubts the presence of an inappropriate sleep environment, and acquires data from a measuring device installed in the bedroom or on the bedding or worn on the user's body. If data cannot be obtained from the measuring device, the system may automatically generate questions based on the following items. The more items that apply, the stronger the system's final determination that “there is a suspicion that the sleep problem is caused by inappropriate temperature, humidity, air flow, noise, vibration, bedding, or other sleep environments.
The temperature in the bedroom is generally outside the range of 22 to 28° C. at bedtime.
The humidity in the bedroom is generally outside the range of 40 to 70% at bedtime.
An air flow with a speed of approximately 0.1 m/s or more is present in the bedroom at bedtime.
The air flow in the bedroom is disturbed or changes over time at bedtime.
A fan is used at bedtime, or an air conditioner is used close to the user's face or body.
Noise levels in the bedroom are generally 30 dB or higher at bedtime.
Perceptible levels of vibration or low-frequency sound are present at bedtime.
The bedding is uncomfortable due to extremely poor ventilation or hardness.
   <n. Respiratory Disorders During Sleep, Such as Apnea while Sleeping, Hypopnea, and Upper Airway Syndrome>

When the user has difficulty maintaining sleep, suffers from excessive daytime sleepiness, or uses sleeping pills or other sedating drugs or substances, the system makes a provisional determination that “there is a suspicion that the sleep problem is caused by respiratory disorders during sleep, such as apnea while sleeping, hypopnea, and upper airway syndrome”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by respiratory disorders during sleep, such as apnea while sleeping, hypopnea, and upper airway syndrome”.
Whether the STOP-BANG questionnaire (Reference: Chung, Frances, Hairil R. Abdullah, and Pu Liao. “STOP-Bang questionnaire including a practical approach to screen for obstructive sleep apnea.” Chest 149.3 (2016): 631-638), which includes ‘snoring, fatigue, witnessed breathing interruptions, hypertension, obesity, age over 50 years, neck circumference over 40 cm, and male gender’, applies to the user or not.
Whether the Berlin Questionnaire (Reference: Netzer, Nikolaus C., et al. “Using the Berlin Questionnaire to identify patients at risk for the sleep apnea syndrome,” J Annals of internal medicine 131.7 (1999): 485-491), which includes ‘snoring, sleepiness or fatigue upon awakening, obesity, or presence of hypertension’, applies to the user or not.
Whether the user's weight has increased by 15 kg or more compared to their weight at the age of 18 years or not
Whether the user's mouth is dry upon waking up or not
Whether the user has an urge to urinate when waking up in the middle of sleep or not
Whether the user has nasal obstruction or enlarged tonsils or not
Whether the user's pillow is too high (generally higher than 5 cm) or not

Instead of the questions described above, when there are data measured by a measuring device or biochemical indices contained in a database in advance, the data or indices may be acquired and used as an aid for the final determination. When the measured data contains a larger number of characteristics below, there is a suspicion that the sleep problem is caused by respiratory disorders during sleep, such as apnea while sleeping, hypopnea, and upper airway syndrome.
Snoring sound is frequently detected.
Sympathetic nerve activity frequently and rapidly increases relative to parasympathetic nerve activity (LF/HF ratio increases) during sleep.
Percutaneous arterial blood oxygen saturation (SpO₂) drops by 3% or more from baseline at least five times per hour.
There is a tendency toward polycythemia (high hemoglobin level, hematocrit level, and red blood cell count).
   <o. Nasal Obstruction, Nasal Discharge, Pain, or Itching>

When the user has difficulty falling asleep or difficulty maintaining sleep or suffers from excessive daytime sleepiness, the system makes a provisional determination that “there is a suspicion that the sleep problem is caused by nasal obstruction, nasal discharge, pain, or itching”. To check whether the provisional determination is correct or not, the system accesses a database that stores the user's medical information, such as receipts, checks for a history of allergic or painful diseases, and displays whether the history exists or the following questions on the user's terminal, and when the system receives more positive responses to the following questions, the system makes a final determination that “there is a stronger suspicion that the sleep problem is caused by nasal obstruction, nasal discharge, pain, or itching”.
The user tends to have a stuffy or runny nose.
The user has had difficulty sleeping due to nasal congestion.
The user perceives nasal congestion in some cases when the user wakes up in the middle of sleep.
The user has an atopic constitution or is prone to dermatitis or itching.
The user sometimes perceives itchiness when the user wakes up in the middle of sleep.
The user is sometimes observed scratching the skin during sleep.
The user does not clean his/her room or does not regularly clean and select bedding, sheets, and nightclothes.
The user sometimes has some pain when going to bed.
The user sometimes perceives pain when waking up in the middle of sleep.
   <p. Iron Deficiency>

When the user has difficulty falling asleep or difficulty maintaining sleep, suffers from excessive daytime sleepiness, or uses sleeping pills or other sedating drugs or substances, the system makes a provisional determination that “there is a suspicion that the sleep problem is caused by iron deficiency”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by iron deficiency”.
Whether the user consumes iron-containing foods or supplements infrequently or not
Whether the user uses iron-free or stainless-steel cooking utensils or not.
When the user is female, whether she has heavy menstrual flow or not.
Whether the user is prone to shortness of breath when climbing stairs or running, and whether the pulse rate rises easily or not.
Whether the user's eyelid conjunctiva is pale red or deathly pale or not.
Whether the user has a craving for ice or not.

Instead of the questions described above, the system may acquire age/gender data, sleep indicators, and biochemical indicators contained in a database in advance and use them as aids for the final determination. When the system receives a larger number of characteristics below, the system makes a final determination that “there is a stronger suspicion that the sleep problem is caused by iron deficiency”.
The user is a woman under 50 years of age.
The level of serum iron or TSAT (transferrin saturation) is low.
The Ferritin value is smaller than 75 ng/ml.
There is a tendency toward anemia (low hemoglobin and hematocrit values).
The red blood cells tend to be microcytic and hypochromic (low MCV, MCH, MCHC).
The pulse rate increases when the amount of activity measured by an acceleration sensor or any other device increases.
There is suspicion of restless legs syndrome/periodic limb movement disorder (will be described later in detail).
   <q. Dietary Habits (Such as Vegetable and Fish Consumption Status and Regularity of Meal Times)>

When the user has difficulty falling asleep or staying asleep, suffers from excessive daytime sleepiness, or uses sleeping pills or other sedating drugs or substances, the system makes a provisional determination that “there is a suspicion that the sleep problem is caused by dietary habits (such as vegetable and fish consumption status and regularity of meal times)”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by dietary habits (such as vegetable and fish consumption status and regularity of meal times)”.
Whether the consumption of vegetables, seaweeds, and mushrooms is small or not.
Whether the consumption of fish is small or not.
Whether the consumption of protein-containing foods is small or not.
Whether the consumption of foods with a high GI value is large or not.
Whether the consumption of probiotics (such as fermented foods and lactic fermenting beverages) is small or not.
Whether the consumption of foods containing minerals such as zinc, magnesium, and calcium is small or not.
Whether the frequency of missing breakfast, lunch, or dinner is high or not.
Whether the period between breakfast and dinner is longer than 12 hours (or the fasting period is shorter than 12 hours) or not.
Whether the period between breakfast and dinner is longer than 16 hours (or the fasting period is shorter than 8 hours) or not.
Whether the user frequently eats immediately before going to bed or when waking up in the middle of sleep or not.
Whether the user eats irregularly or not.
   <r. Inappropriate Bathing Habits>

When the user has difficulty falling asleep or staying asleep, the system makes a provisional determination that “there is a suspicion that the sleep problem is caused by inappropriate bathing habits”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by inappropriate bathing habits”.
The user does not have bathing habits at night, or the user only take a shower but does not soak in the bathtub.
The user takes a bath within two hours before going to bed.
The user takes a bath irregularly every day.
   <s. Inappropriate Exercise Habits>

When the user has difficulty falling asleep or difficulty maintaining sleep, suffers from excessive daytime sleepiness, or uses sleeping pills, the system makes a provisional determination that “there is a suspicion that the sleep problem is caused by inappropriate exercise habits”. To check whether the provisional determination is correct or not, the system makes a final determination that “there is a strong suspicion that the sleep problem is caused by inappropriate exercise habits” based on the user's exercise data via a meter or a wearable terminal or input data on the user's exercise period and when the exercise period does not fall within an appropriate range.

The inventors' research has revealed that the exercise period that minimizes the risk of insomnia is 30.9 hours per week (95% confidence interval: 18.4 to 95.6 hours) in accordance with calculation using a multiple regression model with a quadratic equation (Table 1 below). Similar periods have also been obtained for depression (24.8 hours) and anxiety (state anxiety: 20.8 hours, trait anxiety: 21.2 hours).

Therefore, regardless of the presence or absence of current sleep problems, it is clear that when a measuring device that records and displays the user's total weekly exercise period shows that the total weekly exercise period is significantly shorter than 20 to 31 hours, the exercise period is too short from the viewpoint of psychosomatic effects and sleep, and conversely, when the total exercise time is significantly longer than 20 to 31 hours, the exercise period is too long from the viewpoint of psychosomatic effects and sleep.

TABLE 1

| | | | | | | 95% confidence interval | |
|---|---|---|---|---|---|---|---|
| | Non-standardized coefficient | | Standardized | | | of B | |
| | | Standard | coefficient | | Significance | Lower | Upper |
| | B | error | β | t value | probability | limit | limit |
| (Constant) | 20.027 | .363 | | 55.113 | .000 | 19.313 | 20.741 |
| Total exercise | −.15902 | .04848 | −.37802 | −3.28038 | .00110 | −.25424 | −.06381 |
| duration | | | | | | | |
| (hours/week) | | | | | | | |
| Total exercise | .00257 | .00089 | .33435 | 2.90142 | .00386 | .00083 | .00432 |
| duration | | | | | | | |
| (hours/week)² | | | | | | | |

a. Dependent variable FIRST

<t. Restless Legs Syndrome/Periodic Limb Movement Disorder>

When the user has difficulty falling asleep or staying asleep, suffers from excessive daytime sleepiness, or uses sleeping pills or other sedating drugs or substances, the system makes a provisional determination that “there is a suspicion that the sleep problem is caused by restless legs syndrome/periodic limb movement disorder”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by restless legs syndrome/periodic limb movement disorder”.
The user feels unpleasant sensations, such as pain or restlessness, in the body, especially in the legs, and have a desire to move them.
The unpleasant sensations occur or worsen when the user takes some rest.
The abnormal sensations improve by the user's movement of the legs.
The abnormal sensations occur or worsen in the evening or at night.
The user feels heavy or fatigue sensations in the lower back or the limbs when waking up in the middle of sleep or when waking up in the morning.
The user is pregnant.
The user has an iron deficiency.
The user consumes a large amount of caffeine.
The user has low back pain or spinal cord diseases.
The user uses antidepressants.
The user uses drugs that act as histamine receptor antagonists.
   <u. REM Sleep Behavior Disorder>

When the user has difficulty maintaining sleep, or suffers from excessive daytime sleepiness, the system makes a provisional determination that “there is a suspicion that the sleep problem is caused by REM sleep behavior disorder”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by REM sleep behavior disorder”.
Whether the RBDSQ (Reference: Stiasny-Kolster, Karin, et al. “The REM sleep behavior disorder screening questionnaire-a new diagnostic instrument.” Movement disorders 22.16 (2007): 2386-2393, Reference: Miyamoto, Tomoyuki, et al. “The REM sleep behavior disorder screening questionnaire: validation study of a Japanese version.” Sleep medicine, 10. 10 (2009): 1151-1154), which is a known questionnaire and includes questions such as “dreaming, body movements and talking during sleep, and dropping things around the bed”, applies to the sleep problem or not.
The user frequently talks in his/her sleep.
The user frequently falls out of bed or is about to fall out of bed.
The user sometimes moves his/her arms or legs while sleeping.
The user sometimes frequently dreams of fighting with or being chased by something.

When the user uses a measuring device worn on the human body, a measuring device installed on bedding, or a video monitoring system, and when the measuring device detects behaviors such as violent limb movements or shouting while the user is lying down instead of the questions described above, the system may make a final determination that “there is a strong suspicion that the sleep problem is caused by REM sleep behavior disorder”.

<v. Nocturnal Eating Syndrome, Sleep-Related Eating Disorder, or Nocturnal Hypoglycemia>

When the user has difficulty maintaining sleep, the system makes a provisional determination that “there is a suspicion that the sleep problem is caused by nocturnal eating syndromes, sleep-related eating disorders, or nocturnal hypoglycemia”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by nocturnal eating syndromes, sleep-related eating disorders, or nocturnal hypoglycemia”.
The user sometimes wakes up after falling asleep and eat something, or there is evidence that the user has eaten something.
The user sometimes wakes up during sleep due to hunger or a feeling of having to eat something.
The user unable to resist eating consumes large amounts of foods and drinks before going to bed.
The amount of daily energy consumed from foods and drinks is smaller than ⅔ of a standard daily energy requirement.
The user doesn't or hardly have dinner.
The user suffers from emaciation.
The user suffers from diabetes or uses diabetes medication.

When data containing recorded biochemical indices are available, other data may be acquired to aid the determination instead of the questions described above. For example, When the system receives a larger number of characteristic data below, the system makes a determination that “there is a stronger suspicion that the sleep problem is caused by nocturnal eating syndromes, sleep-related eating disorders, or nocturnal hypoglycemia”.
The value of HbA1c or glycohemoglobin is low.
The value of fasting blood glucose is low.
   <w. Teeth Grinding or Clenching>

When the user has difficulty maintaining sleep or suffers from excessive daytime sleepiness, the system makes a provisional determination that “there is a suspicion that the sleep problem is caused by teeth grinding or clenching”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by teeth grinding or clenching”.
The user is aware of or is pointed out by others that the user grinds or clenches his/her teeth during sleep.
The user sometimes has a painful or fatigued jaw or teeth when waking up.
The user has worn, cracked, or chipped teeth.
The user's parents sometimes grind or clench their teeth during sleep.

Instead of the questions described above, a measuring device worn on the human body may be used to detect persistent overactivity in a jaw electromyogram, or a voice recording device may be used to record teeth grinding sounds to make a final determination.

<x. Central Hypersomnia Such as Narcolepsy>

When the user has excessive daytime sleepiness, the system makes a provisional determination that “there is a suspicion that the sleep problem is caused by central hypersomnia such as narcolepsy. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion that the sleep problem is caused by central hypersomnia such as narcolepsy”.
The contents of responses to questions using Ullanlinna Narcolepsy Scale (Reference: Hublin, C. F. A. U. K., et al. “The Ullanlinna Narcolepsy Scale: validation of a measure of symptoms in the narcoleptic syndrome.” Journal of sleep research 3.1 (1994): 52-59) or Swiss Narcolepsy Scale (Reference: Sturzenegger, Christian, and Claudio L. Bassetti, “The clinical spectrum of narcolepsy with cataplexy: a reappraisal.” Journal of sleep research 13.4 (2004): 395-406), which are each an existing Narcolepsy questionnaire
The user frequently experiences sleep paralysis (night hag).
The user sees hallucinations when falling asleep or napping, or has a dream mixed into consciousness (hallucination on falling asleep).
When the user has aroused emotions or a good time, the user sometimes has difficulty having strength in the body, or becomes weak and fall down (cataplexy).
The user spontaneously wakes up without using an alarm or any other tool and takes a daytime nap even on days when the user has slept for a sufficient amount of time.

When the user has responded to a question “Do you always feel sleepy during the daytime without much sleepiness fluctuation, or do you have sleepiness fluctuations, that is, do you feel not so sleepy during the daytime for a certain period and then suddenly feel unbearable sleepiness in a cycle of about 1.5 hours?” and when the user's response to the question is the latter, the system determines that “there is a stronger suspicion that the sleep problem is caused by narcolepsy”.

<y. Mood, Anxiety, or Psychotic Disorder>

When the user has difficulty falling asleep or difficulty maintaining sleep, or uses sleeping pills or other sedating drugs or substances, the system makes a provisional determination that “there is a suspicion that the sleep problem is caused by mood, anxiety, or psychotic disorder”. To check whether the provisional determination is correct or not, the system automatically generates the following questions and prompts the user to respond to them, or displays from the beginning the questions on the user terminal and prompts the user to respond to them. When the system receives more positive responses to the following questions, the system makes a final determination that “there is a stronger suspicion of mood, anxiety, or psychotic disorder”.
The user makes positive responses to questionnaires related to known mood disorders (such as PHQ, CES-D, QIDS, HAM-D, SDS, MADRS, Depression Dual Questionnaire, YMRS, BDRS, and MES).
The user makes positive responses to known anxiety questionnaires (such as LSAS and STAI).
The user makes positive responses to questionnaires about known psychotic disorders (such as PANSS and PRIME).
The user is unable or unwilling to engage in hobbies or activities that the user used to enjoy.
The user suddenly feels very anxious, experiences distress, or starts to hyperventilate.
The user sometimes feels as if something very terrible is going to happen.
The user feels like he/she wants to die or has to die.
The user feels as if his/her thoughts are communicated or read by someone else.
The user becomes very sensitive to noise or vibration and feels pain.

### E: Means for Prioritizing and Creating Methods for Addressing Finally Determined Causes

The system prioritizes the lifestyle items that the user should improve for the type and cause of the user's sleep problem having been finally determined (finalized and identified) by the means of C and D described above, and either sends the result to the user terminal and/or a terminal for the administrator, or prints the result out on a paper medium.

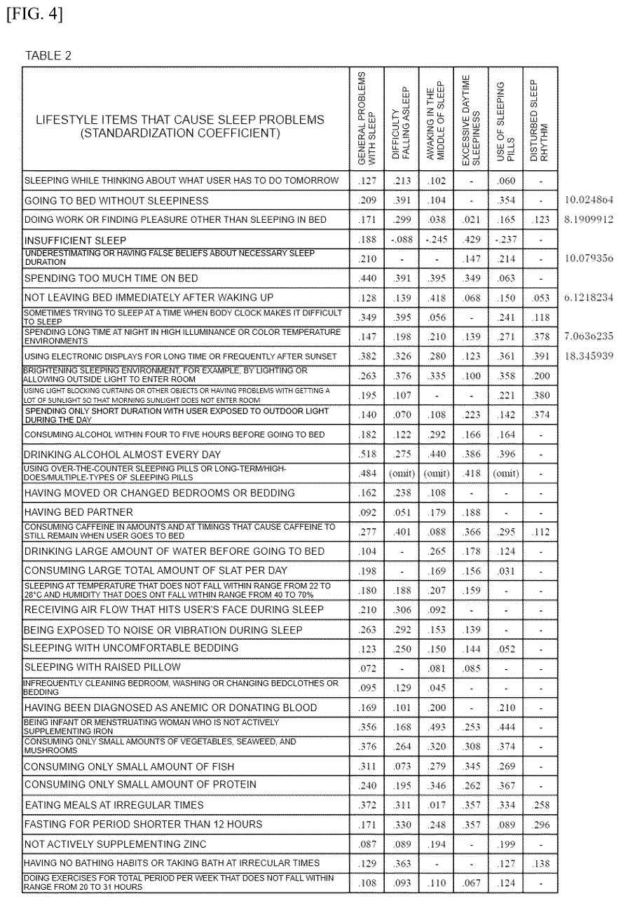

The lifestyle items to be improved are prioritized and displayed or printed out, and the prioritization process uses Table 2 shown in FIG. 4. The “standardized coefficients of the influence of the lifestyles on the sleep problems” shown in Table 2 have been revealed by the past research conducted by the inventors.

Table 2 shows unique coefficients (odds ratio/adjusted odds ratio) that give rise to each of the sleep problems, and some of the coefficients are displayed after simplified for illustrative purposes. For example, the explanatory variable is derived by a multivariate regression analysis using categorical data converted into a dummy variable, but in practice, the sleep duration, exercise period, integral illuminance data, and other pieces of information can be used in the form of continuous variables. Also, the objective variable is analyzed by using a continuous variable of a score measured in an existing sleep scale, but in practice, the objective variable may be categorical data having about two or three values representing whether it occurs or not.

The coefficients are also shown as the standardized coefficients, but may in practice be non-standardized coefficients, for example, the effect on the number of awakenings in the middle of sleep depending on the choice of a variable, or may instead be odds ratios in multivariate logistic regression analysis for analyzing whether the sleep problems occur or not, or may still instead be coefficients for prediction obtained by machine learning. The items themselves are also variable.

The system determines, based on the coefficients, that the user (or administrator) can improve the sleep problems to be improved by preferentially addressing the lifestyle items thought to have a significant impact on the sleep problems in descending order of the coefficients. As a result of weighting based on the coefficients, the contents of the lifestyle items to be improved are prioritized and presented to the user and/or the administrator.

Based on the above, the system presents to the user and/or the administrator the following lifestyle improvement measures, based on the finally identified causes and Table 3, which is not shown.

(a) Examples Presented when the Identified Causes are “Problems with the Circadian Rhythm Due to the Body Clock (Delayed, Non-24, Irregular Rhythm)”
Do not go to bed at a time when your body clock makes it difficult to sleep.
Shorten the amount of time spent in high illuminance or color temperature environments at night.
Do not use electronic displays for a long time or frequently after sunset.
Do not brighten your sleeping environment, for example, by lighting or allowing outside light to enter the room.
Do not use light blocking curtains or other objects to allow morning sunlight to enter the room.
   (b) Examples Presented when the Identified Causes are “Problems with the Circadian Rhythm Due to the Body Clock (Advanced)”
Lengthen the amount of time spent in high illuminance or color temperature environments at night.
Reduce the frequency of exposure to morning sunlight.
   (c) Examples Presented when the Identified Causes are “Anxiety, Hyperarousal, or Conditioning (Psychophysiological Insomnia)”
Do not go to bed thinking about what you have to do tomorrow; instead, make a to-do list or something similar before going to sleep.
Do not go to bed when not being sleepy.
Do not engage in activities or entertainments in the bed other than sleeping.
Keep on-bed time short.
Get out of bed as soon as possible after waking up.
   (d) Examples Presented when the Identified Causes are “Insufficient Sleep/Sleep Debt
Get enough sleep.
Do not underestimate or have false beliefs about the required sleep duration.
   (e) Examples Presented when the Identified Causes are “Excessively Long On-Bed Periods or Inappropriate Bedtimes”
Keep on-bed time short.
Get out of bed immediately after waking up.
Do not go to bed at a time when your body clock makes it difficult to sleep.
   (f) Examples Presented when the Identified Causes are “Inappropriate Nighttime Light Exposure”
Shorten the amount of time spent in high illuminance or color temperature environments at night.
Do not use electronic displays for a long time or frequently after sunset.
Do not brighten your sleeping environment, for example, by lighting or allowing outside light to enter the room.
   (g) Examples Presented when the Identified Causes are “Insufficient Morning and Daytime Light Exposure”
Do not use light blocking curtains or other objects to get a lot of sunlight and allow morning sunlight to enter the room.
Lengthen the period of exposure to outdoor light in a day.
   (h) Examples Presented when the Identified Causes are “Inappropriate Alcohol Consumption”
Do not consume alcohol within four to five hours before going to bed.
Do not drink alcohol almost every day.
   (i) Examples Presented when the Identified Causes are “Inappropriate Use of Sleeping Pills or Other Sedative Drugs or Substances”
Do not use over-the-counter sleeping pills.
Do not use long-term, high-dose, or multiple-dose (multiple-kind) sleeping pills.
   (j) Examples Presented when the Identified Causes are “Sudden Change in Sleeping Environment”
The user seems to suffer from temporary insomnia due, for example, to a change of residence or a change of bedroom/bedding. The sleep state may improve as the user gets used to the new environment, but care should be taken to avoid a shift to conditioned insomnia. When the inability to sleep well persists, see a medical professional.
   (k) Examples Presented when the Identified Causes are “Inappropriate Caffeine Consumption”
Do not consume caffeine in amounts and at timings that cause the caffeine to still remain when the user goes to bed.
   (l) Examples presented when the identified causes are “inappropriate water and salt consumption”
Do not drink a large amount of water before going to bed.
Do not consume any kind of soups, such as soup, miso soup, or noodles, for dinner.
Reduce the total daily salt consumption.
   (m) Examples Presented when the Identified Causes are “Inappropriate Temperature, Humidity, Air Flow, Noise, Vibration, Bedding, or Other Sleeping Environment”
Keep the temperature around the body and face between 22 to 28° C. and the humidity between 40 to 70% when going to bed.
Do not let an air flow hit the user's face when going to bed.
Eliminate noise and vibration when going to bed.
Use comfortable bedding.
Do not raise the pillow too high.
Clean the bedroom and wash/change bedclothes and bedding more often.
Do not have other people (bed partners or pets) in the same bedding.
   (n) Examples Presented when the Identified Causes are “Respiratory Disorders During Sleep, Such as Apnea while Sleeping, Hypopnea, and Upper Airway Syndrome”
Resolve obesity.
Do not gain 5 to 15 kg or more in weight as compared with the weight at the time when the user was 18 to 20 years old.
Do not raise the pillow too high.
   (o) Examples Presented when the Identified Causes are “Nasal Obstruction, Nasal Discharge, Pain, or Itching”
Clean the bedroom and wash/change bedclothes and bedding more often.
   (p) Examples Presented when the Identified Causes are “Iron Deficiency”
When the user suffers from anemia, improve the anemia.
Refrain from red blood cell donation when symptoms persist.
When the user is an infant or a menstruating woman, actively supplement iron.
   (q) Examples Presented when the Identified Causes are “Dietary Habits (Such as Vegetable and Fish Consumption Status and Regularity of Meal Times)”
Increase consumption of vegetables, seaweeds, and mushrooms.
Increase fish consumption.
Increase protein consumption.
Keep regular meal times.
Make fasting fall within 12 hours.
Increase zinc consumption.
   (r) Examples Presented when the Identified Causes are “Inappropriate Bathing Habits”
Make it a habit to take a bath.
Take a bath at regular times.
   (s) Examples Presented when the Identified Causes are “Inappropriate Exercise Habits”
Make it a habit to exercise.
Ensure that the total weekly exercise time falls within 20 to 31 hours.
   (t) Examples Presented when the Identified Causes are “Restless Legs Syndrome/Periodic Limb Movement Disorder”
Increase iron consumption.
Refrain from consuming caffeine in amounts and at timings that cause it remain when going to bed.
   (u) Examples Presented when the Identified Causes are “REM Sleep Behavior Disorder”

When the cause described above causes the sleep problem, there is no corresponding lifestyle and no presentation is made. However, a text that encourages the user to seek medical attention may be displayed, as will be described later.

(v) Examples Presented when the Identified Causes are “Nocturnal Eating Syndrome, Sleep-Related Eating Disorder, or Nocturnal Hypoglycemia”
Keep regular meal times.
Eat dinner every time.
Do not restrict your diet excessively.
Do not go to bed at a time when your body clock makes it difficult to sleep.
Shorten the amount of time spent in high illuminance or color temperature environments at night.
Do not use electronic displays for a long time or frequently after sunset.
Do not brighten your sleeping environment, for example, by lighting or allowing outside light to enter the room.
Do not use light blocking curtains or other objects to allow morning sunlight to enter the room.
   (w) Examples Presented when the Identified Causes are “Teeth Grinding or Clenching”
Increase iron consumption.
Do not consume caffeine in amounts and at timings that cause the caffeine to still remain when the user goes to bed.
Do not raise the pillow too high.
   (x) Examples Presented when the Identified Causes are “Central Hypersomnia Such as Narcolepsy”

When the cause described above causes the sleep problem, there is no corresponding lifestyle and no presentation is made. However, a text that encourages the user to seek medical attention may be displayed, as will be described later.

(y) Examples Presented when the Identified Causes are “Mood, Anxiety, or Psychotic Disorder”

When the cause described above causes the sleep problem, there is no corresponding lifestyle and no presentation is made. However, a text that encourages the user to seek medical attention may be displayed, as will be described later.

The system may output the results described above as a summary shown in Table 2 below in the form of a text to the administrator. The summary includes the characteristics of the user's sleep problems, the current sleep pattern, the current sleep status, suspicion of a disease to be treated at a medical institution, and lifestyle problems that need to be improved. The output allows the administrator to easily cite or transcribe how the system is used, for example, in his/her own medical records.

TABLE 2
Chief complaints) difficulty in falling asleep, awakenings in the middle of
sleep, and excessive daytime sleepiness
Sleep schedule) holidays wake up at 3:00, fall asleep at 4:00, wake up at
11:00
Differential diagnosis) #Circadian rhythm sleep-wake disorders,
#Abuse/dependence on hypnotic/sedative
Lifestyles to be improved) nighttime excessive light exposure, use of
displays before going to bed, and excessive caffeine

When the system determines that the sleep problems cannot be resolved by the “lifestyle improvements” described above and there is a disease-related problem, the system can determine that there is a strong suspicion of a sleep disorder requiring treatment, and can also present the determination to the user and/or the administrator. At this time, a “medical information form or a draft thereof” may be issued automatically. At that time, information about the user's name, age, place of residence, occupation, medical history, family history, life history, current medication status, and onset of the sleep problems may be appended if available, or, when no such information is available, the user may be prompted to enter it via the present system.

### <Circadian Rhythm Sleep-Wake Disorders

When the system detects “a problem with the circadian rhythm due to the body clock (delayed, non-24, irregular rhythm)”, “a problem with the circadian rhythm due to the body clock (advanced)”, or “a problem caused by shift work or suspicion of a shift work sleep disorder”, the system assumes that there is a suspicion of circadian rhythm sleep-wake disorders and records at least the daily sleep schedule (time when user goes to bed and time when user wakes up), and outputs information, if available, on the duration of the problem or the time when the problem occurred and obtained additional responses to the questions generated by the system to encourage examination and treatment as required at a medical institution.

### <Alcohol Dependence>

When the system detects a sleep problem “due to inappropriate alcohol consumption”, and receives a large number of positive responses to the questions generated by the system, the system assumes that there is a suspicion of “alcohol dependence,” and outputs information, if available, on the duration of the problem or the time when the problem occurred and obtained additional responses to the questions generated by the system to encourage examination and treatment as required at a medical institution.

### <Abuse of and Dependence on Hypnotic/Sedative Drugs>

When the system detects a sleep problem “due to abuse of and dependence on hypnotic and sedative drugs”, the system assumes that there is a suspicion of “abuse of and dependence on hypnotic/sedative drugs”, and outputs information, if available, on the duration of the problem or the time when the problem occurred and obtained additional responses to the questions generated by the system to encourage examination and treatment if necessary as required institution.

### <Sleep Disordered Breathing>

When the system detects a sleep problem “due to respiratory disorders during sleep, such as apnea while sleeping, hypopnea, and upper airway syndrome”, the system assumes that there is a suspicion of “sleep disordered breathing”, and outputs information, if available, on the duration of the problem or the time when the problem occurred and the contents of obtained additional responses to the questions generated by the system and the status of biochemical indices and device data contained in the database to encourage examination and treatment as required at a medical institution.

### <Rhinitis and Nasal Obstruction>

When the system detects a sleep problem “due to nasal obstruction, nasal discharge, pain, or itching”, and receives a large number of positive responses to additional questions regarding nasal obstruction and nasal discharge, the system assumes that there is a suspicion of “rhinitis/nasal obstruction”, and outputs the suspicion along with additional response data. At this time, the system outputs a text stating “suspicion of allergic rhinitis” and “suspicion of inferior nasal concha hypertrophy and nasal septal deviation” at the same time as differentially diagnosed diseases to encourage examination and treatment as required at a medical institution.

### <Dermatitis>

When the system detects a sleep problem “due to nasal obstruction, nasal discharge, pain, or itching”, and receives a large number of positive responses to additional questions regarding itching, the system assumes that there is a suspicion of “dermatitis”, and outputs the suspicion along with additional response data. At this time, the system outputs a text stating “suspicion of allergic dermatitis” at the same time as a differentially diagnosed disease to encourage examination and treatment as required at a medical institution.

### <Restless Legs Syndrome/Periodic Limb Movement Disorder>

When the system detects a sleep problem “due to restless legs syndrome/periodic limb movement disorder”, the system assumes that there is a suspicion of “restless legs syndrome/periodic limb movement disorder”, and outputs information, if available, on the duration of the problem or the time when the problem occurred and the contents of obtained additional responses to the questions generated by the system to encourage examination and treatment as required at a medical institution.

### <REM Sleep Behavior Disorder>

When the system detects a sleep problem “due to REM sleep behavior disorder”, and receives a large number of positive responses to the questions generated by the system, the system assumes that there is a suspicion of “REM sleep behavior disorder”, and outputs information, if available, on the duration of the problem or the time when the problem occurred and obtained additional responses to the questions generated by the system to encourage examination and treatment as required at a medical institution.

### <Nocturnal Eating Syndrome/Sleep-Related Eating Disorder>

When the system detects a sleep problem “due to nocturnal eating syndrome, sleep-related eating disorder, or nocturnal hypoglycemia”, the system assumes that there is a suspicion of “nocturnal eating syndrome/sleep-related eating disorder”, and outputs information, if available, on the duration of the problem or the time when the problem occurred and obtained additional responses to the questions generated by the system to encourage examination and treatment as required at a medical institution.

### <Teeth Grinding and Bruxism During Sleep>

When the system detects a sleep problem “due to teeth grinding or clenching”, the system assumes that there is a suspicion of “teeth grinding and bruxism during sleep”, and outputs the suspicion along with additional response data to encourage examination and treatment as required at a medical institution.

### <Central Hypersomnia>

When the system detects a sleep problem “due to central hypersomnia such as narcolepsy”, and receives a large number of positive responses to the questions generated by the system, the system assumes that there is a suspicion of “central hypersomnia”, and outputs information, if available, on the duration of the problem or the time when the problem occurred and obtained additional responses to the questions generated by the system to encourage examination and treatment as required at a medical institution. When the response to a question about the presence or absence of weakness during an emotional outburst is positive, the system may output a suspected disease name of “narcolepsy with cataplexy” or “narcolepsy type 1”. When the responses to the additional questions indicate a strong suspicion of idiopathic hypersomnia, the system may note the strong suspicion, and when the responses indicate a strong suspicion of narcolepsy, the system may note the strong suspicion.

### <Mood, Anxiety, or Psychotic Disorder>

When the system detects a sleep problem “due to mood, anxiety, or psychotic disorder”, the system assumes that there is a suspicion of “mental disorder”, and outputs information, if available, on the duration of the problem or the time when the problem occurred and obtained additional responses to the questions generated by the system. At this time, when the system receives a large number of positive responses to a questionnaire on known mood disorders, the system outputs a suspected name of “depression”, when the system receives a large number of positive responses to a questionnaire on known anxiety, the system outputs a suspected name of an “anxiety disorder”, and when the system receives a large number of positive responses to a questionnaire on known psychotic disorders, the system outputs a suspected name of a “psychotic disorder” to encourage examination and treatment as required at a medical institution.

### Weighted Presentation in the Case of Paper-Based Medical Questions

When the user is asked to respond to a questionnaire on a non-digital medium such as paper, the system can score in the following manner to determine the presence or absence and the degree of the current sleep problems and future risks.

The system has the user answer whether the questionnaire includes at least one of the following items: “Sleeping while thinking about what the user has to do tomorrow, going to bed without sleepiness, doing work or finding a pleasure other than sleeping in bed, insufficient sleep, underestimating or having false beliefs about the necessary sleep duration, spending too much time on the bed, not leaving the bed immediately after waking up, sometimes trying to sleep at a time when the body clock makes it difficult to sleep, spending a long time at night in high illuminance or color temperature environments, using electronic displays for a long time or frequently after sunset, brightening the sleeping environment, for example, by lighting or allowing outside light to enter the room, using light blocking curtains or other objects or having problems with getting a lot of sunlight so that morning sunlight does not enter the room, spending only a short period with the user exposed to outdoor light during the day, consuming alcohol within four to five hours before going to bed, drinking alcohol almost every day, using over-the-counter medications having hypnotic effects, using long-term/high-dose/multiple-types of sleeping pills, having moved or changed bedrooms or bedding, having a bed partner, consuming caffeine in amounts and at timings that cause the caffeine to still remain when the user goes to bed, drinking a large amount of water before going to bed, consuming a large total amount of salt per day, sleeping at a temperature that does not fall within the range from 22 to 28° C. and a humidity that does not fall within the range from 40 to 70%, receiving an air flow that hits the user's face during sleep, being exposed to noise or vibration during sleep, sleeping with uncomfortable bedding, sleeping with raised pillow, infrequently cleaning the bedroom, washing or changing bedclothes or bedding, having been diagnosed as anemic or donating blood, being an infant or a menstruating woman who is not actively supplementing iron, consuming only small amounts of vegetables, seaweed, and mushrooms, consuming only small amount of fish, consuming only small amount of protein, eating meals at irregular times, fasting for a period shorter than 12 hours, not actively supplementing with zinc, having no bathing habits or taking a bath at irregular times, and doing exercises for a total period per week that does not fall within a range from 20 to 31 hours.” In addition to the items described above, the system may have the user record his/her age and gender and when the sleep problems began if the user are experiencing the problems.

The user records in the questionnaire a coefficient corresponding to each of the items described above, and after responding to the questionnaire, the respondent or scorer adds up all the values, and applies the result of the addition to a regression equation representing the relationship with the sleep problems to determine the presence or absence and the degree of the sleep problems at present and future risks. At this point, since the calculation becomes complicated when the values have decimal points, the result is multiplied by an arbitrary coefficient and rounded off to a nearest whole number, which reduces the accuracy of the result but reduces the burden on the person who does the addition. Furthermore, since the calculation becomes complicated when the values have negative coefficients, arbitrary numbers are added to the values to provide positive numbers, and then the sum of the arbitrary numbers are subtracted in the final tally, so that the burden on the person who does the addition is reduced. When the coefficients are derived from linear regression, the sum of the regression coefficients corresponding to the items selected above is a continuous variable that predicts the degree and intensity of the following items: “general problems with sleep”; “difficulty falling asleep”; “difficulty maintaining sleep”; “excessive daytime sleepiness”; “use of sleeping pills or other sedative drugs or substances”; and “problems with sleep rhythm”, and can determine whether or not the user currently has a sleep problem, if so, to what extent, and whether or not the user is at risk of developing a sleep problem in the future.

In the case of a logit model based on multivariate logistic regression analysis, in which the coefficients are not linear, the sum of the regression coefficients corresponding to the items selected above can be logit-transformed or otherwise processed to show the possibility of whether or not the user has “general problems with sleep”, “difficulty falling asleep”, “difficulty maintaining sleep”, “excessive daytime sleepiness”, “use of sleeping pills or other sedative drugs or substances”, and “sleep rhythm problems”. In this process, in determination of the risk based on the final total score, since applying the logit model formula is complicated and makes extremely difficult it to do the calculation on the spot, the resultant total score simplifies the determination by creating a correspondence table showing whether or not the user currently has various sleep problems, the degree of the problems, and future risks.

In addition, on the paper to be viewed by the user, in addition to or in place of the coefficients described above, rank ratings such as A, B, C, D, E, etc. can be added in descending order to make it easier to understand the priorities in accordance with which the problematic items described above need to be improved. The user can grasp the presence or absence and the degree of sleep problems in the current situation and future risks from the rank ratings, and the user can be clearly informed that the checked items are items that need to be improved in accordance with the priorities, A, B, C, D, E, etc.

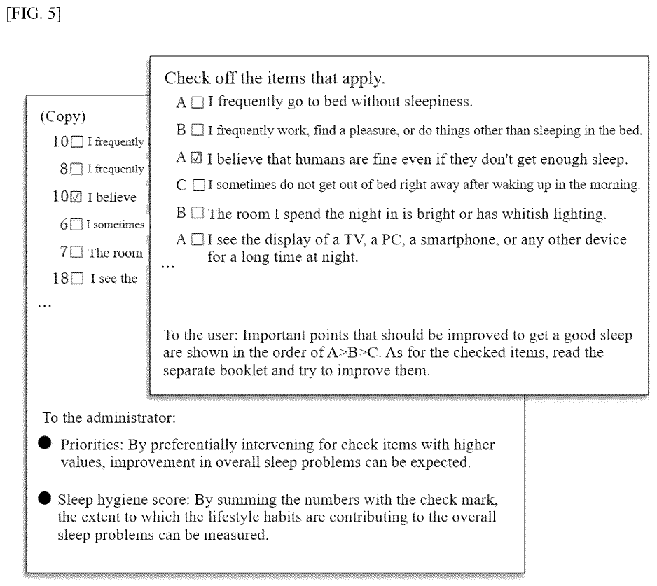

In addition, in the case of paper media, the results can be copied so that the user can take them home and use them as materials for the improvement, and the administrator can also keep the records. FIG. 5 shows an example of implementing the above.

### Commitment Registration and Follow-Up Reminders

Since mere provision of information alone is uncertain to be effective in encouraging the user to change his/her behavior, the system may encourage the user to register “commitments” as to which and how the user is going to deal with the lifestyle improvement items presented to them. The system may also allow the administrator to view the registered commitment information. The system may allow the user and the administrator to check the commitments at any time via the user and administrator terminals, and may send a notification to the user and administrator terminals as a follow-up reminder to allow the user and the administrator to check whether the user is working on the improvements. The notification can be sent two to three days, one week, or two weeks after the day after the commitments are registered, with reference to a learning curve, to enhance effectiveness of the commitments.

### Provided via API

The system may be provided with an API on an online server to call the series of processes described above from other systems on the network. Upon reception of a series of data inputs, the API transmits, as output information in response to the received data, at least one of the following pieces of information: the contents of additional question items, causes of suspected sleep problems, remedies or weighted remedies, lectures or other contents given with the aid of documents, images, and videos tagged to the remedies or weighted remedies, and the scored sleep state to the user terminal and/or the administrator terminal, and displays the sent information on the user terminal and/or administrator terminal screen.

F: Means for Recalculating and Creating Weighting of Addressing Methods in Accordance with Specified Items or Individual Analysis Groups

As described above, the present system includes the means A to E and desirably further includes “F: means for recalculating and creating weighting of addressing methods in accordance with specified items or individual analysis groups” as means for modifying “E: Means for prioritizing and creating a method for addressing the finally determined cause” as appropriate.

As described above, the present system handles diverse question items, and especially when the user has multiple sleep problems overlapping with each other, the number of items to be responded by the user is enormous. The tendency is even more pronounced in the case of paper-based questions that cannot be successively generated by electromagnetic methods. This may prevent the user from easily responding to the questions. On the other hand, there is a possibility that new kinds of lifestyle that affect human sleep will appear in the future.

In view of the circumstances described above, the system may allow the administrator to select items that he/she wants the user to respond and/or to specify new items. At this time, the system refers to the existing database and performs regression analysis or machine learning using the items specified by the administrator as explanatory variables and various scores or degrees of the following items as objective variables: “general sleep problems”; “difficulty falling asleep”; “difficulty maintaining sleep”; “excessive daytime sleepiness”; “use of sleeping pills or other sedating drugs or substances”; and “sleep rhythm problems”, outputs and stores coefficients for limited or newly specified question items, and can use the coefficients for weighting and prioritizing remedies for the limited or newly specified question items.

### Successive Recalculation System

The impact of sleep hygiene (lifestyle) described above on sleep and its coefficients may vary on a population group basis. For example, the elderly tend to have less adverse effects of inappropriate light exposure at night on sleep due to decreased light sensitivity, and the Japanese tend to have more adverse effects of alcohol on sleep due to lower alcohol decomposition. There are also racial differences in caffeine metabolism.

In view of the circumstances described above, when the present system is used in any individual population, the system may refer to a database containing data obtained for that population, and use successively accumulated lifestyle-related responses as the explanatory variables, and perform regression analysis or machine learning on the various scores or degrees of the following items as the objective variables: “general sleep problems”; “difficulty falling asleep”; “difficulty maintaining sleep”; “excessive daytime sleepiness”; “use of sleeping pills or other sedating drugs or substances”; and “sleep rhythm problems”, which are obtained on the spot or after a certain period of time in response to the responses, then recalculate the coefficients, and update the values tagged to the system or printed on a questionnaire sheet.

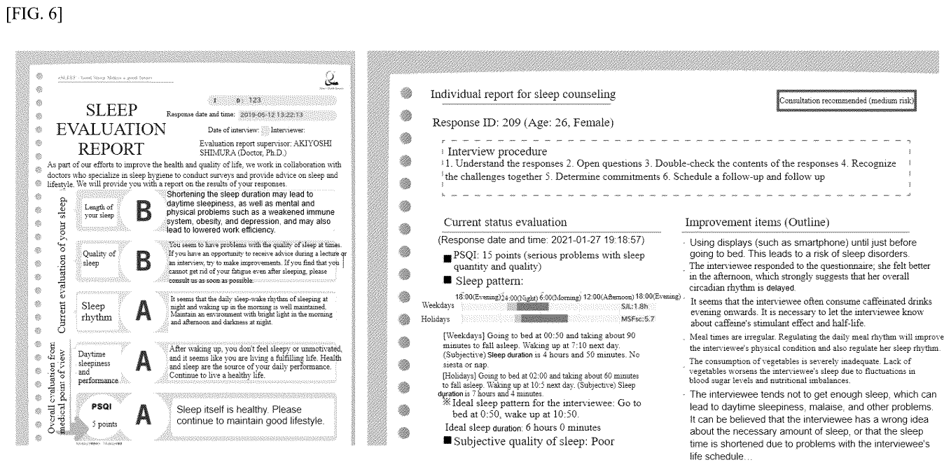

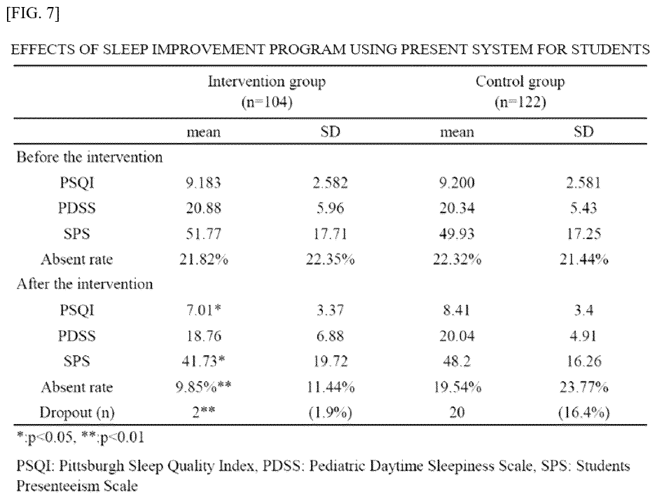

FIG. 6 shows an example of an evaluation report to be submitted (output) to the user and/or the administrator. FIG. 7 shows the results of a randomized controlled trial (RCT) given to students in the Tokyo metropolitan area. The comparison test was given on 226 subjects, and shows that 104 students who used the present system to present remedies for intervention improved their PSQI scores representing the following items as compared with 122 students who did nothing: the general sleep problems were improved by 23.8%; the daytime sleepiness was improved by 10.2%; the performance was improved by 19.4%; and the dropout rate was improved by 88.3%.

In an experiment conducted on general workers for a simple intervention, PSQI scores representing the general sleep problems were improved by 14.9%, difficulty falling asleep improved by 27.5%, awakening in the middle of sleep improved by 18.9%, and the productivity improved by 7.1%.

What is claimed:

1. A method for assisting in remedying a sleep problem of a user comprising the steps of:
(i) monitoring a sleep duration and time zone of a day by displaying a questionnaire to the user on the user terminal and recording a result of a response to the questionnaire in a first database; by using a device that is worn by the user and measures a sleep state of the user, and recording a result of the measurement in the first database; by using a device that is attached to bedding and measures the sleep state of the user, and recording a result of the measurement in the first database; or by using a device that measures the sleep state of the user by using a video system, and recording a result of the measurement in the first database;
(ii) monitoring sleep quality and a daytime function by displaying a questionnaire to the user on the user terminal and recording a result of a response to the questionnaire in a second database; by using a device that is worn by the user and measures the sleep state of the user, and recording a result of the measurement in the second database; by using a device that is attached to the bedding and measures the sleep state of the user, and recording a result of the measurement in the second database; and by using a device that measures the sleep state of the user by using a video system, and recording a result of the measurement in the second database;
(iii) identifying whether the sleep problem of the user is (a) a difficulty type in falling asleep, (b) a difficulty type in maintaining sleep, (c) an excessive daytime sleepiness type, or (d) other type, based on information recorded in the first and second databases in steps (i) and (ii) and by using predetermined criteria;
(iv) selecting candidate causes of the type of sleep problem identified in step (iii), from a first table created in advance that lists probable causes of each of the difficulty type in falling asleep, the difficulty type in maintaining sleep, the excessive daytime sleepiness type, and the other type;
(v) making a final determination of causes of the sleep problem by making a final determination of whether each of the candidate causes selected in step (iv) is true or not based on a result of response to additionally given lifestyle questions, using predetermined criteria, and
(vi) prioritizing countermeasures for the causes finally determined in step (v) by prioritizing lifestyle items selected as applicable in the lifestyle questions used in step (v), as lifestyle items to be improved, by looking up standardized coefficients in a second table created in advance showing standardized coefficients of an impact of lifestyle items corresponding to the lifestyle questions used in step (v), which standardized coefficients are determined in advance separately for each type of sleep problem described in step (iii), wherein the standardized coefficients corresponding to the selected lifestyle items are obtained for the type of sleep problem identified in step (iii), and countermeasures corresponding to the selected lifestyle items are prioritized in descending order of the standardized coefficients, and
(vii) providing the countermeasures prioritized in step (vi) to a user terminal and/or an administrator terminal, thereby assisting in remedying the sleep problem of the user.

2. The method according to claim 1, wherein, in step (iii), (d) other type includes a case where the user suffers from the sleep disorder that requires medical treatment, and step (iii) is performed based not only on the information recorded in the databases A and B and also on predefined information on a sleep disorder that requires medical treatment, wherein when the case is identified as the sleep problem in step (iii), in step (vii), information requiring diagnosis of the user's disorders and/or a draft of information submission form for medical treatment is provided as the countermeasures to the user terminal and/or the administrator terminal.

3. The method according to claim 1, wherein, in step (vii), the countermeasures includes at least one of the following pieces of information as output information: contents of additional question items, causes of suspected sleep problems, remedies or weighted remedies, lectures or other contents given with the aid of documents, images, and videos tagged to the remedies or the weighted remedies, and a scored sleep state.
